(19) United States
(12) Patent Application Publication
(10) Pub. No.: US 2023/0212667 A1
(43) Pub. Date: Jul. 6, 2023

(54) METHODS OF NUCLEIC ACID SEQUENCING USING SURFACE-BOUND PRIMERS

(71) Applicants: ILLUMINA CAMBRIDGE LIMITED, CAMBRIDGE (GB); ILLUMINA, INC., SAN DIEGO, CA (US)

(72) Inventors: Jonathan Mark Boutell, Cambridge (GB); Eli M. Carrami, Cambridge (GB); Pietro Gatti Lafranconi, Cambridge (GB); Philip Balding, Cambridge (GB); Oliver Jon Miller, Cambridge (GB); Kay Klausing, San Diego, CA (US); Justin Robbins, San Diego, CA (US); Xiaolin Wu, Cambridge (GB)

(73) Assignees: ILLUMINA CAMBRIDGE LIMITED, CAMBRIDGE (GB); ILLUMINA, INC., SAN DIEGO, CA (US)

(21) Appl. No.: 18/089,867

(22) Filed: Dec. 28, 2022

Related U.S. Application Data
(60) Provisional application No. 63/294,622, filed on Dec. 29, 2021.
(60) Provisional application No. 63/408,026, filed on Sep. 19, 2022.

(51) Int. Cl. C12Q 1/6874

(52) U.S. Cl. CPC C12Q 1/6874; C12Q 1/6806

(57) ABSTRACT

Polynucleotide sequencing methods for sequencing one or more polynucleotide templates use primers bound to a surface as sequencing primers. The surface primers may include at least a portion of a surface oligonucleotide used during cluster formation. The sequencing methods may be used for single stranded sequencing or double stranded sequencing. Double stranded sequencing methods may employ an enzyme that has nick-translation activity. A kit includes all the reagents needed for sequencing does not include sequencing primers. The kit may be used to accomplish the sequencing methods of the present disclosure.

### CROSS REFERENCE TO RELATED APPLICATIONS

This application claims the benefit of U.S. Provisional Pat. Application No. 63/294,622, filed December 29^{th}, 2021 and U.S. Provisional Pat. Application No. 63/408,026, filed September 19^{th}, 2022, each of which are incorporated herein by reference in its entirety The present disclosure relates to, among other things, sequencing of polynucleotides.

### SEQUENCE LISTING

This application contains a Sequence Listing electronically submitted to the United States Patent and Trademark Office via Patent Center as an XML file entitled “0531.002259WO01” having a size of 9.04 kilobytes and created on December 22^{nd}, 2022. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD

The present disclosure relates to, among other things, sequencing of polynucleotides.

### INTRODUCTION

Improvements in sequencing methodologies have allowed for sequencing of pooled or multiplexed polynucleotides from different libraries in a single sequencing protocol. A library-specific sequence (an “index tag”) may be added to polynucleotides of each library so that the origin of each sequenced polynucleotide may be properly identified. The index tag sequence may be added to polynucleotides of a library by, for example, ligating adapters comprising the index tag sequence to ends of the polynucleotides.

The adapters may contain sequences in addition to the index tag sequence, such as a universal extension primer sequence and a universal sequencing primer sequence. The universal extension primer sequence may, among other things, hybridize to a first oligonucleotide coupled to a solid surface. The first oligonucleotide may have a free 3′ end from which a polymerase may add nucleotides to extend the sequence using the hybridized library polynucleotide as a template, resulting in a reverse strand of the library polynucleotide being coupled to the solid surface. Additional copies of forward and reverse strands may be coupled to the solid surface through cluster amplification. One example of cluster amplification is bridge amplification in which the 3′ end of previously amplified polynucleotides that are bound to the solid surface hybridize to second oligonucleotides bound to the solid surface. The second oligonucleotide may have a free 3′ end from which a polymerase may add nucleotides to extend the sequence using the coupled reverse strand polynucleotide as a template, resulting in a forward strand of the library polynucleotide being coupled to the solid surface via the second oligonucleotide. The process may be repeated to produce clusters of forward and reverse strands coupled to the solid surface. The forward strands or the reverse strands may be removed, e.g. via cleavage, prior to sequencing.

Each polynucleotide bound to the solid support includes a target nucleic acid sequence for which the identity of the nucleotides making up that sequence is desired and one or more index sequences that are used for determining the source from which the target nucleotide was isolated. In traditional next-generation sequencing techniques, separate sequencing primers are needed to read each index sequence and to read the target nucleic acid sequence. For example, for single read sequencing of a polynucleotide that has one index sequence, two sequencing primers are needed, an index primer and a target nucleic acid primer. For paired-end sequencing of a polynucleotide that has one index region, three sequencing primers are needed, an index primer, and two target nucleic acid primers. As the number of desired index sequence reads grows, the number of sequencing primers increases. For example, for paired-end sequencing of a polynucleotide that includes two index sequences, four sequencing primers are needed, two index primers, and two target nucleic acid primers.

Next-generation sequencing equipment includes draws reagents from premade cartridges. Separate cartridges are needed for each sequencing step. For example, to accomplish sequencing one index sequence read and one target nucleic acid read, two cartridges are needed each containing the appropriate sequencing primer and other sequencing components. Thus, as the number of desired sequence reads per polynucleotide increase, the number of primers and cartridges increases.

It would be desirable to reduce the number of reagents and cartridges used during sequencing to, for example, reduce material consumption, consumer and manufacturing costs, and manufacturing complexity for next-generation sequencing platforms while maintaining high data quality.

### SUMMARY

Presented herein, among other things, are methods for sequencing one or more polynucleotide templates using oligonucleotide primers that are attached to a solid surface (e.g., surface primers). In embodiments, the surface primers comprise at least a portion of the surface oligonucleotides that are used during cluster formation.

In one aspect, the present disclosure describes a method for sequencing a polynucleotide template. The method includes (a) providing a surface, a first surface oligonucleotide, a second surface primer, and a first polynucleotide template. The first surface oligonucleotide and the second surface primer are bound to the surface at their respective 5′ ends. The first polynucleotide template is covalently bound to the 3′ end of the first surface oligonucleotide and has a free 3′ end. The second surface primer has a free 3′ end that is hybridized to at least a portion of the 3′ end of the first template polynucleotide. The method further includes (b) sequencing at least a portion of the first polynucleotide template by extending the second surface primer from the free 3′ end thereby generating a second polynucleotide template that includes a first read region. The first polynucleotide template is used as a template and at least a portion of the second surface primer is used as a primer. The second polynucleotide template is covalently bound to the surface primer and has a free 3′ end. The second polynucleotide template is complementary to the first polynucleotide template and complementary to at least a portion of the first surface oligonucleotide in proximity to the free 3′ end. The method further includes (c) cleaving the first surface oligonucleotide or a 5′ portion of the first polynucleotide template to produce a first surface primer and a cleaved first polynucleotide. The first surface primer is bound to the surface at the 5′ end and has a free 3′ end. The cleaved first polynucleotide has a free 5′ end and a free 3′ end. The method further includes (d) sequencing at least a portion of the second polynucleotide template by extending the first surface primer from the free 3′ end thereby generating a third polynucleotide template that includes a second read region. The second polynucleotide template is used as a template and at least a portion of the first surface primer is used as a primer.

In some embodiments, step (a) further includes providing a fourth polynucleotide template complementary to the first polynucleotide template that is covalently bound to the 3′ end of the second surface oligonucleotide. The fourth polynucleotide template comprises a free 3′ end. At least a portion of the fourth polynucleotide template in proximity to the free 3′ end is hybridized to at least a portion of the first surface oligonucleotide. The method further includes cleaving the second surface oligonucleotide or a 5′ portion of the fourth polynucleotide template to produce the second surface primer and a cleaved fourth polynucleotide template having a free 5′ end and a free 3′ end.

In some embodiments the method of cleaving the second surface oligonucleotide or a 5′ portion of the fourth polynucleotide template further includes removing a first excisable base to generate a cleaved second surface oligonucleotide. The method may further include generating a hydroxyl at the free 3′ end of the cleaved second surface oligonucleotide to give the second surface primer.

In some embodiments, the method further includes providing a cleaved fourth polynucleotide template have a free 5′ end and a free 3′ end, wherein the cleaved fourth polynucleotide template is hybridized to at least a portion of the first polynucleotide template.

In some embodiments, the extension of the second surface primer from the free 3′ end during sequencing of at least the portion of the first polynucleotide template results in displacement of at least a 5′ portion of the cleaved fourth polynucleotide template from the first polynucleotide template.

In some embodiments, sequencing at least a portion of the first polynucleotide template further includes removing nucleotides and/or polynucleotides from the cleaved fourth polynucleotide template thereby shortening the cleaved fourth polynucleotide template. In some embodiments, the nucleotides and/or polynucleotides are removed by an enzyme or a fusion protein that has nick translation activity.

In some embodiments the method further includes denaturing the cleaved fourth polynucleotide template from the first polynucleotide template and washing the surface to remove the cleaved fourth polynucleotide template prior to sequencing at least the portion of the first polynucleotide template.

In some embodiments the method of cleaving the first surface oligonucleotide or a 5′ portion of the first polynucleotide template further includes removing a second excisable base to generate a cleaved first surface oligonucleotide. The method may further include generating a hydroxyl at the free 3′ end of the cleaved first surface oligonucleotide to give the first surface primer.

In some embodiments, the method further includes denaturing the cleaved first polynucleotide template from the third polynucleotide template and washing the surface to remove the cleaved first polynucleotide template prior to sequencing at least a portion of the second polynucleotide template.

In another aspect, the present disclosure describes a kit. The kit includes all reagents that are needed for sequencing at least the portion the first polynucleotide template and at least the portion of the second polynucleotide template. The kit may be free of sequencing primers. The kit may include sequencing primers.

The details of one or more embodiments are set forth in the accompanying drawings and the description below. Other features, objects, and advantages will be apparent from the description and drawings, and from the claims.

It is to be understood that both the foregoing general description and the following detailed description present embodiments of the subject matter of the present disclosure and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF DRAWINGS

The following detailed description of specific embodiments of the present disclosure may be best understood when read in conjunction with the following drawings.

FIG. 1 is a flow diagram illustrating an overview of a sequencing method consistent with the sequencing methods of the present disclosure.

FIG. 2 is a flow diagram illustrating an overview of a pre-sequencing method consistent with embodiments disclosed herein.

FIGS. 3A and 3B are schematic drawings illustrating a first sequencing workflow consistent with embodiments disclosed herein.

FIG. 4 is a schematic drawing illustrating a second sequencing workflow consistent with embodiments disclosed herein.

FIG. 5 is a schematic drawing illustrating a third sequencing workflow consistent with embodiments disclosed herein.

FIG. 6 is a schematic drawing illustrating a pre-sequencing workflow consistent with embodiments disclosed herein

FIG. 7A and FIG. 7B are plots showing the data intensity by cycle and Q score distribution, respectively, from an example sequencing run where the index sequences and the universal sequences were skipped using dark cycling.

FIG. 8A and FIG. 8B are plots showing the data intensity by cycle and Q score distribution, respectively FIGS. 8A and 8B show data of an example sequencing run consistent with embodiments disclosed herein where the index sequences and the target nucleic acid were sequenced, and dark cycling was used to skip the universal sequences.

FIGS. 9A and 9B show results from the sequencing run of FIG. 8. FIG. 9A shows the Q30 by cycle plots for the indexing runs and FIG. 9B shows the demultiplexing results.

FIGS. 10A and 10B shows plots illustrating the sequencing error rate of double stranded surface sequencing via nick translation with two different enzymes compared to a double stranded surface sequencing via strand displacement control. SBM is scan mix, CBM is cleavage mix, and IBM is incorporation mix.

FIG. 11 is a bar graph showing the error rate over G-quadraplex sequences when using single strand surface sequencing, double strand surface sequencing via strand displacement, and double strand sequencing via nick translation.

FIG. 12 is a plot showing the difference in sequencing signal intensity of single strand surface sequencing and double strand surface sequencing.

FIGS. 13A-B are example synthetic schemes showing a cleavage reaction at the allyl-T of an oligonucleotide using Pd(0) (A) and OsO₄ (B).

FIG. 14A is a schematic drawing illustrating an ffC incorporation assay.

FIG. 14B is a plot showing fluorescence intensity over time using a ffC incorporation assay with Pol(X)and various probes.

FIG. 15A is a schematic drawing of an assay used to generate the results shown in FIG. 15B.

FIG. 15B shows images of agar gels following gel electrophoresis of polynucleotides resulting from the assay illustrated in FIG. 15A various polymerase-flap nuclease constructs.

FIG. 16A is a schematic drawing of an assay used to generate the results shown in FIG. 15B.

FIG. 16B shows images of gels following gel electrophoresis of polynucleotides resulting from the assay illustrated in FIG. 15A comparing various polymerase-flap nuclease constructs.

FIGS. 17A(1), 17A(2) and 17A(3) are plot showing the time to incorporate 50% of a template using various polymerase-flap nuclease constructs.

FIGS. 17B-C are plots showing fluorescence intensity over time using a ffC incorporation assay with Pol 1901 (FIG. 17B) and GAK Helix Pol (FIG. 17C) and various probes.

FIGS. 18A-B are plots showing phasing weight (A) and error rate (B) using a GAN Helix-Pol(X) polymerase-flap nuclease construct.

FIGS. 18C-18D are images of gels following gel electrophoresis of polynucleotides resulting from a flap cleavage assay using GAN only (18A) and a GAN-Helix-Pol(A) fusion construct (18D).

FIGS. 19A-B are images of gels following gel electrophoresis of polynucleotides resulting from a flap cleavage assay using GAN only (19A) and a GAN-Pol(X) (19B) fusion linked with a TAQ linker.

FIG. 20 is a plot of error rate per sequencing cycle for various polymerases, polymerase-flap nuclease combinations and polymerase-flap nuclease constructs.

FIG. 21 is a plot created using the Integrative Genomics Viewer from the Broad Institute comparing errors with single stranded and double stranded sequencing at a region of the human genome containing a G-Quadruplex (G-quad).

FIG. 22 is a plot of signal lost during a sequencing run vs laser dosage, comparing single and double stranded sequencing.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. Thus, for example, reference to a “template polynucleotide sequence” includes examples having two or more such “template polynucleotide sequences” unless the context clearly indicates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

As used herein, “have”, “has”, “having”, “include”, “includes”, “including”, “comprise”, “comprises”, “comprising” or the like are used in their open-ended inclusive sense, and generally mean “include, but not limited to”, “includes, but not limited to”, or “including, but not limited to”.

“Optional” or “optionally” means that the subsequently described event, circumstance, or component, can or cannot occur, and that the description includes instances where the event, circumstance, or component, occurs and instances where it does not.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising an incorporation step, a detection step, a deprotection step, and one or more wash steps includes embodiments where the method consists of enumerated steps and embodiments where the method consists essentially of the enumerated.

As used herein, “providing” in the context of a compound, composition, or article means making the compound, composition, or article, purchasing the compound, composition or article, or otherwise obtaining the compound, composition or article.

As used herein, the term “chain extending enzyme” is an enzyme that produces a copy replicate of a polynucleotide using the polynucleotide as a template strand. For example, the chain extending enzyme may be an enzyme having polymerase activity. Typically, DNA polymerases bind to the template strand and then move down the template strand sequentially adding nucleotides to the free hydroxyl group at the 3′ end of a growing strand of nucleic acid. DNA polymerases typically synthesize complementary DNA molecules from DNA templates and RNA polymerases typically synthesize RNA molecules from DNA templates (transcription). The polymerase may be linked to another protein or domain of a protein such as, for example, a flap nuclease. Polymerases may use a short RNA or DNA strand, called a primer, to begin strand growth. Some polymerases may displace the strand upstream of the site where they are adding bases to a chain. Such polymerases are said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity. Any suitable polymerase may be used with the methods and/or compositions (e.g., kits) or the present disclosure. In some embodiments, the polymerase is a polymerase described in U.S. Provisional Pat. Application No. 63/412,241, U.S. Pat. Application No. US16/703569 (US11001816B2), PCT Application Number PCT/US2013/03169 (WO2014142921A1) all of which are hereby incorporated by reference in its entirety.

The terms “polynucleotide” and “oligonucleotide” are used interchangeably herein to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). As used herein, “amplified target sequences” and its derivatives, refers generally to a polynucleotide sequence produced by the amplifying the target sequences using target-specific primers and the methods provided herein. The amplified target sequences may be either of the same sense (e.g., the positive strand) or antisense (i.e., the negative strand) with respect to the target sequences.

The term “polynucleotide template” or “template polynucleotide” refer to a polymeric form of a nucleotide that includes a target nucleic acid and an adaptor on one or both ends.

Suitable nucleotides for use in the provided methods include, but are not limited to, deoxynucleotide triphosphates, deoxyadenosine triphosphate (dATP), deoxythymidine triphosphate (dTTP), deoxycytidine triphosphate (dCTP), and deoxyguanosine triphosphate (dGTP). Optionally, the nucleotides used in the provided methods, whether labeled or unlabeled, can include a blocking moiety such as a reversible terminator moiety that inhibits chain extension. Suitable labels for use on the labeled nucleotides include, but are not limited to, haptens, radionucleotides, enzymes, fluorescent labels, chemiluminescent labels, and chromogenic agents.

A polynucleotide will generally contain phosphodiester bonds, although in some cases nucleic acid analogs can have alternate backbones, comprising, for example, phosphoramidite (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y.S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y.S. Sanghui and P. Dan Cook. Polynucleotides containing one or more carbocyclic sugars are also included within the definition of polynucleotides (see Jenkins et al., Chem. Soc. Rev. (1995) pg. 169-176). Several polynucleotide analogs are described in Rawls, C & E News Jun. 2, 1997 page 35. All these references are hereby expressly incorporated by reference. These modifications of the ribose-phosphate backbone may be done to facilitate the addition of labels, or to increase the stability and half-life of such molecules in physiological environments.

A polynucleotide will generally contain a specific sequence of four nucleotide bases: adenine (A); cytosine (C); guanine (G); and thymine (T). Uracil (U) can also be present, for example, as a natural replacement for thymine when the nucleic acid is RNA. Uracil can also be used in DNA (dU). A polynucleotide may also include native or non-native bases. In this regard, a native deoxyribonucleic acid polynucleotide may have one or more bases selected from the group consisting of adenine, thymine, cytosine, or guanine and a ribonucleic acid may have one or more bases selected from the group consisting of uracil, adenine, cytosine, or guanine. It will be understood that a deoxyribonucleic acid polynucleotide used in the methods or compositions set forth herein may include, for example, uracil bases and a ribonucleic acid can include, for example, a thymine base. Exemplary non-native bases that may be included in a nucleic acid, whether having a native backbone or analog structure, include, without limitation, inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. Optionally, isocytosine and isoguanine may be included in a nucleic acid in order to reduce non-specific hybridization, as generally described in U.S. Pat. No. 5,681,702, which is incorporated by reference herein in its entirety.

A non-native base used in a polynucleotide may have universal base pairing activity such that it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which base pairs with cytosine, adenine or uracil.

Incorporation of a nucleotide into a polynucleotide strand refers to joining of the nucleotide to a free 3′ hydroxyl group of the polynucleotide strand via formation of a phosphodiester linkage with the 5′ phosphate group of the nucleotide. The polynucleotide template to be sequenced can be DNA or RNA, or even a hybrid molecule that includes both deoxynucleotides and ribonucleotides. The polynucleotide can include naturally occurring and/or non-naturally occurring nucleotides and natural or non-natural backbone linkages.

The terms “primer oligonucleotide”, “oligonucleotide primer”, and “primer” are used throughout interchangeably and are polynucleotide sequences that are capable of annealing specifically to one or more polynucleotide templates to be amplified or sequenced. Generally, primer oligonucleotides are single-stranded or partially single-stranded. Primers may also contain a mixture of non-natural bases, non-nucleotide chemical modifications or non-natural backbone linkages so long as the non-natural entities do not interfere with the function of the primer. Typically, the primer functions as a substrate onto which nucleotides may be polymerized by a polymerase; in some embodiments, however, the primer may become incorporated into the synthesized polynucleotide strand and provide a site to which another primer may hybridize to prime synthesis of a new strand that is complementary to the synthesized nucleic acid molecule. The primer may include any combination of nucleotides or analogs thereof. In some embodiments, the primer is a single-stranded oligonucleotide or polynucleotide.

As used herein, the term “double stranded,” when used in reference to a nucleic acid molecule, means that substantially all of the nucleotides in the nucleic acid molecule are hydrogen bonded to a complementary nucleotide. A partially double stranded nucleic acid can have at least 10%, at least 25%, at least 50%, at least 60%, at least 70%, at least 80%, at least 90% or at least 95% of its nucleotides hydrogen bonded to a complementary nucleotide.

As defined herein, “sample” and its derivatives is used in its broadest sense and includes any specimen, culture and the like that is suspected of including a target nucleic acid. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual; a collection of nucleic acid samples from genetically related members; nucleic acid samples from genetically unrelated members; nucleic acid samples (matched) from a single individual such as a tumor sample and normal tissue sample; or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

As used herein, the term “adapter” and its derivatives, e.g., universal adapter, refers generally to any linear oligonucleotide which can be ligated to a target nucleic acid. In some embodiments, the adapter is substantially non-complementary to the 3′ end or the 5′ end of any target sequence present in a sample. In some embodiments, suitable adapter lengths are in the range of about 10-100 nucleotides, about 12-60 nucleotides and about 15-50 nucleotides in length. Generally, the adapter can include any combination of nucleotides and/or nucleic acids. In some embodiments, the adapter can include one or more cleavable groups at one or more locations. In some embodiments , the adapter can include a sequence that is substantially identical, or substantially complementary, to at least a portion of a primer. In some embodiments , the adapter can include a sequence that is substantially identical, or substantially complementary, to at least a portion of a surface oligonucleotide. In some embodiments, the adapter can include a barcode, also referred to as an index or tag, to assist with downstream error correction, identification, or sequencing. The terms “adaptor” and “adapter” are used interchangeably.

The term “ally-dNTP,” such as ally-thymine (ally-T), ally-cytosine (ally-C), ally-guanine (ally-G), and ally-adenine (ally-A) refer to a nucleotide that has an ally group at the 5′ carbon of the ribose or deoxyribose sugar. An ally-dNTP can be incorporated at any point in an oligonucleotide or nucleic acid. An example structure of a dinucleotide that includes an ally-T is shown below.

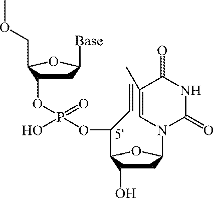

The term “surface oligonucleotide” refers to a polymeric form of a nucleotide that is attached to a surface. In some embodiments, the surface oligonucleotide is attached through the surface at the 5′ end and has a free 3′ end. The terms “P5” (SEQ ID NO: 1), “P7” (SEQ ID NO: 2), “P15” (SEQ ID NO: 3), and “P17” (SEQ ID NO: 4) may be used when referring to a surface oligonucleotide. P5, P7, P15, and P17 are described in U.S. Pat. Pub. No. US 2019/0352327. The terms “P5’” (P5 prime),“P7’” (P7 prime), “P15′” (P15 prime), and “P17′” (P17 prime) refer to the complement of P5, P7, P15, and P17 respectively. It will be understood that any suitable surface oligonucleotide can be used in the methods presented herein, and that the use of P5, P7, P15, and P17 are exemplary embodiments only. Uses of surface oligonucleotide such as P5, P7,P15, P17 on flowcells is known in the art, as exemplified by the disclosures of WO 2007/010251, WO 2006/064199, WO 2005/065814, WO 2015/106941, WO 1998/044151, and WO 2000/018957. In some embodiments, the surface oligonucleotide or at least a portion of the surface oligonucleotide may function as a surface primer for sequencing. In view of the general knowledge available and the teachings of the present disclosure, one of skill in the art will understand how to design and use sequences that are suitable for surface oligonucleotides and surface primers for sequencing.

As used herein, the term “universal sequence” refers to a region of sequence that is common to two or more target nucleic acids, where the molecules also have regions of sequence that differ from each other. A universal sequence that is present in different members of a collection of molecules can allow capture of multiple different nucleic acids using a population of capture nucleic acids that are complementary to a portion of the universal sequence, e.g., a universal capture binding sequence. Non-limiting examples of universal capture binding sequences include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal primer binding site. Target nucleic acid molecules may be modified to attach universal adapters (also referred to herein as adapters), for example, at one or both ends of the different target sequences, as described herein.

As used herein, the term “different,” when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different from each other while also having a universal sequence region that are the same as each other.

As used herein, the term “nucleic acid” is intended to be consistent with its use in the art and includes naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from adenine, thymine, cytosine or guanine and a ribonucleic acid can have one or more bases selected from uracil, adenine, cytosine or guanine. Useful non-native bases that can be included in a nucleic acid are known in the art. The term “target,” when used in reference to a nucleic acid (e.g, “nucleic acid target” or “target nucleic acid”) is intended as a semantic identifier for the nucleic acid in the context of a method or composition set forth herein and does not necessarily limit the structure or function of the nucleic acid beyond what is otherwise explicitly indicated. A “target nucleic acid” having an adapter at one or more ends, is referred to as a polynucleotide template.

In addition, the recitations herein of numerical ranges by endpoints include all numbers subsumed within that range (e.g., 1 to 5 includes 1, 1.5, 2, 2.75, 3, 3.80, 4, 5, etc.). Where a range of values is “greater than”, “less than”, etc. a particular value, that value is included within the range.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. However, it will be understood that a presented order is one embodiment of an order by which the method may carried out. Any recited single or multiple feature or aspect in any one claim may be combined or permuted with any other recited feature or aspect in any other claim or claims.

### DETAILED DESCRIPTION

Reference will now be made in greater detail to various embodiments of the subject matter of the present disclosure, some embodiments of which are illustrated in the accompanying drawings.

Presented herein are methods relating to sequencing polynucleotides. Specifically, the present disclosure provides methods for sequencing one or more polynucleotide templates using oligonucleotide primers that are attached to a surface (e.g., surface primers). In some embodiments, the sequencing method using surface primers comprises sequencing a single stranded polynucleotide. In some embodiments, the sequencing method using surface primers comprises sequencing a strand of a double stranded polynucleotide. Sequencing of the strand of the double stranded polynucleotide may proceed via strand displacement, nick translation, or any other suitable mechanism.

In some embodiments, the sequencing methods of the present disclosure are particularly useful for next generation sequencing, also called massively parallel sequencing. Next generation sequencing allows many target nucleic acids (e.g., polynucleotide templates) to be sequenced simultaneously.

Preparation of target nucleic acids for sequencing may include one or more of (i) preparing a library of polynucleotide templates from target nucleic acids, (ii) immobilizing the library of polynucleotide templates onto a surface, and (iii) amplifying the immobilized polynucleotide templates. The amplified polynucleotide templates may be sequenced according to the methods described herein to determine the sequence of at least a portion of the target nucleic acids.

### Preparing a Library of Polynucleotide Templates

Libraries of polynucleotide templates may be prepared in any suitable manner. In embodiments, preparing a library of polynucleotide templates includes obtaining the target nucleic acids and ligating adapters to the target nucleic acids to create polynucleotide templates.

As used herein, the term “target nucleic acid” refers to a nucleic acid molecule where identification of at least a portion of its nucleotide sequence is desired. The target nucleic acid may be essentially any nucleic acid of known or unknown sequence. The sequence of two or more target nucleic acids in the population of target nucleic acids may be the same or different.

Sequencing may result in the determination of the sequence of a part of the target nucleic acid or the entire target nucleic acid. The target nucleic acid or a population of target nucleic acids can be derived from one or more primary nucleic acid samples. A primary nucleic acid sample may originate in double-stranded DNA (dsDNA) form (e.g., genomic DNA fragments, PCR and amplification products, and the like) or may originate in single-stranded form, as DNA or RNA that may been converted to dsDNA.

A primary target nucleic acid may be obtained from any biological sample using known, routine methods. Suitable biological samples include, but are not limited to, a blood sample, biopsy specimen, tissue explant, organ culture, biological fluid, or any other tissue or cell preparation, or fraction thereof, or derivative thereof, or isolated therefrom. In some embodiments, a primary target nucleic acid may be obtained as a sample from a human, an animal, a bacterium, a fungus, or a virus.

The target nucleic acid or a population of target nucleic acids can be derived from a primary nucleic acid sample that has been sequence specifically fragmented or randomly fragmented. For example, a fragment of genomic DNA or cDNA may be used as a target nucleic acid or a population of target nucleic acids. Random fragmentation refers to the fragmentation of a nucleic acid from a primary nucleic acid sample in a non-ordered fashion by enzymatic, chemical, or mechanical methods. Such fragmentation methods are known in the art and use standard methods (e.g., see Sambrook and Russell, Molecular Cloning, A Laboratory Manual, third edition).

Once the target nucleic acid or population of target nucleic acids are obtained, a library of polynucleotide templates for use in the provided sequencing methods may be prepared using a variety of standard techniques available and known in the art. The term “library” refers to the collection of polynucleotide templates containing known common sequences at their 3′ and/or 5′ ends, for example, by attachment of adapters. Each polynucleotide template of the library includes one or more target nucleic acids. Exemplary methods of polynucleotide template preparation include, but are not limited to, those described in Bentley et al., Nature 456:49-51 (2008); U.S. Pat. No. 7,115,400; and U.S. Pat. Application Publication Nos. 2007/0128624; 2009/0226975; 2005/0100900; 2005/0059048; 2007/0110638; and 2007/0128624, each of which is herein incorporated by reference in its entirety.

For the sequencing methods of the present disclosure, the polynucleotide templates include adapters that are ligated to the 5′ and/or 3′ ends of the target nucleic acid. Methods for attaching adapters to one or both ends of a target nucleic acid are known to the person skill in the art. The attachment can be through standard library preparation techniques using, for example, ligation (U.S. Pat. Pub. No. 2018/0305753), or tagmentation using transposase complexes (Gunderson et al., WO 2016/130704).

Adapters include one or more known sequences. When the polynucleotide template includes adapters with known sequences on the 5′ and/or 3′ ends, the known sequences may be the same or different. Consistent with the methods of present disclosure, known adapter sequence located on the 5′ and/or 3′ ends of the polynucleotide templates are capable of hybridizing to one or more surface oligonucleotides that are immobilized on a surface. For instance, for use with a surface that includes P5 and P7 surface oligonucleotides, the adapters may include P5′ or a P7′ sequence or derivative thereof. The P5 surface oligonucleotide may hybridize with the P5′ adapter sequence and the P7 surface oligonucleotide may hybridize with the P7′ adapter sequence. Optionally, polynucleotide templates may include one or more detectable labels. The one or more detectable labels may be attached to the polynucleotide template at the 5′ end, at the 3′ end, and/or at any nucleotide position within the polynucleotide template, for example, within the adapter sequence.

The adapters may further include one or more universal sequences. A universal sequence is a region of nucleotide sequence that is common to, e.g., shared by, two or more polynucleotide templates, where the two or more polynucleotide templates also have regions of sequence differences (e.g., the target nucleic acid). A universal sequence that may be present in different members of a library of polynucleotide templates may allow the replication or amplification of multiple different sequences using a single universal primer that is complementary to the universal sequence. Similarly, at least one, two (e.g., a pair), or more universal sequences that may be present in different members of a library of polynucleotide templates may allow the replication or amplification of multiple different sequences using at least one, two (e.g., a pair), or more single universal primers that are at least partially complementary to the universal sequences. Thus, a universal primer includes a sequence that may hybridize specifically to such a universal sequence.

The adapters may also include one or more index sequences. An index can be used as a marker characteristic of the source of particular target nucleic acid (U.S. Pat. No. 8,053,192). Generally, the index is a synthetic sequence of nucleotides that is part of the adapter which is added to the target nucleic acids as part of the library preparation step. Accordingly, an index is a nucleic acid sequence which is attached to each of the target nucleic acids of a particular sample, the presence of which is indicative of, or is used to identify, the sample or source from which the target nucleic acids were isolated. In some embodiments, a dual index system may be used. In a dual index system, the adapter attached to target nucleic acids includes two different index sequences, for example as described in U.S. Pat. No. 10,975,430; U.S. Pat. No. 10,995,369; U.S. Pat. No. 10,934,584; and U.S. Pat. Pub. No. 2018/0305753.

In some embodiments, the adapters comprise a cleavage site. The adapters may include any suitable cleavage site. Examples of suitable cleavage sites include abasic cleavage sites, chemical cleavage sites, ribonucleotide cleavage sites, photochemical cleavage sites, hemimethylated DNA cleavage sites, nicking endonuclease cleavage sites, and restriction enzyme cleavage sites.

The polynucleotide templates may also be modified to include any nucleic acid sequence desirable using standard, known methods. The modifications may be incorporated as a part of the adapter or separately, for example, prior to adapter ligation. Such additional sequences may include, but are not limited to, restriction enzyme sites, non-natural nucleotides, modified nucleic acids, and combinations thereof. Example of unnatural or modified nucleic acids include, but are not limited to, deoxyuridine (U), 8-oxo-guanine (8-oxo-G), hemimethylated sequences, ally-dNTPs (e.g., ally-T, ally-C, ally-G, and ally-A), and deoxyinosine.

In some embodiments, the polynucleotide templates may include one or more modified nucleotides that enhances base pair binding, relative to a natural nucleotide, to a nucleotide of the template polynucleotide. The modifications may be incorporated as a part of the adapter or separately, for example, prior to adapter ligation. Modified nucleotides are known and include, for example, locked nucleotides (LNAs) and bridged nucleotides (BNAs). LNAs and BNAs, as well as oligonucleotides containing LNAs and BNAs, are commercially available. The following publications provide additional information regarding BNAs: (1) Obika, S., et al., (1997), “Synthesis of 2′-O,4′-C-methyleneuridine and -cytidine. Novel bicyclic nucleosides having a fixed C3, -endo sugar puckering,” Tetrahedron Letters. 38 (50): 8735; (2) Obika, S., et al., (2001), “3′-amino-2’,4′-BNA: Novel bridged nucleic acids having an N3′-->P5′ phosphoramidate linkage,” Chemical communications (Cambridge, England) (19): 1992-1993; (3) Obika, S., et al., (2001), “A 2’,4′-Bridged Nucleic Acid Containing 2-Pyridone as a Nucleobase: Efficient Recognition of a C·G Interruption by Triplex Formation with a Pyrimidine Motif,” Angewandte Chemie International Edition. 40 (11): 2079; (4) Morita, K., et al., (2001), “2′-O,4′-C-ethylene-bridged nucleic acids (ENA) with nuclease-resistance and high affinity for RNA,” Nucleic Acids Research. Supplement. 1 (1): 241-242; (5) Hari, Y., et al., (2003), “Selective recognition of CG interruption by 2’,4′-BNA having 1-isoquinolone as a nucleobase in a pyrimidine motif triplex formation,” Tetrahedron. 59 (27): 5123; (6) Rahman, S. M. A., et al., (2007), “Highly Stable Pyrimidine-Motif Triplex Formation at Physiological pH Values by a Bridged Nucleic Acid Analogue,” Angewandte Chemie International Edition. 46 (23): 4306-4309. LNAs monomers include an additional bridge that connects the 2′ oxygen and the 4′ carbon of a ribose moiety to “lock” the ribose in the 3′-endo conformation. Preferably, the modified nucleotides form standard Watson-Crick base pairs. For example, LNA bases form standard Watson-Crick base pairs but the locked configuration increases the rate and stability of the base pairing (Jepsen et al., Oligonucleotides, 14, 130-146 (2004)).

In some embodiments, the polynucleotide templates may include non-natural backbone linkages such as a diol or disulfide; photo-cleavable spacer group; or any combination thereof. The modifications may be incorporated as a part of the adapter, or separately prior to adapter ligation.

In some embodiments, prior to or after adapter ligation, the polynucleotides templates are amplified. Amplification may be accomplished through any known amplification process known in the art, for example, solid-phase amplification, polony amplification, colony amplification, polymerase chain reaction (PCR) such as emulsion PCR, bead rolling circle amplification (RCA), surface RCA, or surface exponential strand displacement (SDA). Amplification can be thermal or isothermal.

### Immobilization of the Library of Polynucleotide Templates Onto a Surface

As used herein the term surface refers to a substrate for attaching nucleic acids. A surface is made of material that has a rigid or semi-rigid structure to which a polynucleotide can be attached or upon which nucleic acids can be synthesized and/or modified. Surfaces can include any resin, gel, bead, well, column, chip, flow cell, membrane, matrix, plate, filter, glass, controlled pore glass (CPG), polymer support, membrane, paper, plastic, plastic tube or tablet, plastic bead, glass bead, slide, ceramic, silicon chip, multi-well plate, nylon membrane, fiber optic, and PVDF membrane. In some embodiments, the surface is within or a part of a flow cell.

The surface includes a population of surface oligonucleotides that are immobilized on the surface. The surface oligonucleotides may be covalently attached to the surface. The surface oligonucleotides are generally configured to bind or hybridize to a portion of a polynucleotide template, particularly to a portion of the adapter of the polynucleotide template. The surface oligonucleotides are attached to the surface at the 5′ end and have a free 3′ end. The population of surface oligonucleotides may include a population of a first surface oligonucleotide and a population of a second surface oligonucleotide where the first surface oligonucleotide and the second surface oligonucleotide have different sequences. In some embodiments, the first surface oligonucleotide includes the sequences P7 (SEQ ID NO. 1). In some embodiments, the second surface oligonucleotide includes the sequence of P5 (SEQ ID NO. 2). In some embodiments, the second surface oligonucleotide includes the sequence of P15 (SEQ ID NO. 3). The P7, P5, and P15 surface oligonucleotides are configured to hybridize with the P7′, P5′, and P15′ sequences of adapters attached to template polynucleotides. Uses of surface oligonucleotides such as P5 and P7 on flow cells is known in the art, as exemplified by the disclosures of WO 2007/010251, WO 2006/064199, WO 2005/065814, WO 2015/106941, WO 1998/044151, and WO 2000/018957. P7, P5, and P15 surface oligonucleotides are also described in, for example, US 2019/0352327, which is hereby incorporated by reference in its entirety. In some embodiments, additional populations of surface oligonucleotides having sequences different from the first and second surface oligonucleotides may be present. Attachment of the surface oligonucleotides to the surface can be accomplished through any method known in the art, for example, such as those described in U.S. Pat. No. 8,895,249, WO 2008/093098, and U.S. Pat. Pub. No. 2011/0059865 A1, amongst others. In some embodiments, the surface oligonucleotides may include one or more unnatural or modified nucleic acids, unnatural backbone linkages, restriction enzyme sequences, or any combination thereof, such as those described elsewhere herein.

The polynucleotide templates are immobilized on the surface through hybridization of the adapter portion that is configured to bind to at least one surface oligonucleotide. For example, if the population of first surface oligonucleotides includes the P5 sequence, polynucleotide templates that include the P5′ sequence in the adapter region may hybridize to the first surface oligonucleotide. If the population of first surface oligonucleotides includes the P7 sequence, polynucleotide templates that include the P7′ sequence in the adapter region may hybridize to the first surface oligonucleotide. If the population of first surface oligonucleotides includes the P15 sequence, polynucleotide templates that include the P15′ sequence in the adapter region may hybridize to the first surface oligonucleotide.

The surface oligonucleotides may be used as primers for chain extension or amplification using as templates the hybridized polynucleotide templates.

### Surface Amplification of the Polynucleotide Templates

The polynucleotide templates may be amplified on the surface to which they are immobilized. Polynucleotide template amplification includes the process of amplifying or increasing the numbers of a polynucleotide templates and/or of a complement thereof, by producing one or more copies of the template and/or or its complement. Amplification may be carried out by a variety of known methods under conditions including, but not limited to, thermocycling amplification or isothermal amplification. For example, methods for carrying out amplification are described in U.S. Pat. Pub. No. 2009/0226975; WO 98/44151; WO 00/18957; WO 02/46456; WO 06/064199; and WO 07/010251; which are incorporated by reference herein in their entireties.

Briefly, amplification may occur on the surface to which the polynucleotide templates are immobilized. This type of amplification can be referred to as solid phase amplification, which when used in reference to polynucleotide templates, refers to any polynucleotide template amplification reaction carried out on or in association with a surface. Typically, all or a portion of the amplified products are synthesized by extension of a primer that is immobilized on the surface.

Solid-phase amplification may include a polynucleotide template amplification reaction including only one species of surface oligonucleotide immobilized to a surface. Alternatively, the surface may comprise a plurality of first and second different immobilized surface oligonucleotide species. Solid phase polynucleotide template amplification reactions generally include at least one of two different types of nucleic acid amplification, interfacial or surface (or bridge) amplification. For instance, in interfacial amplification the surface includes a polynucleotide template that is indirectly immobilized to the solid support by hybridization to an immobilized surface oligonucleotide, the immobilized surface oligonucleotide may be extended in the course of a polymerase-catalyzed, template-directed elongation reaction (e.g., primer extension) to generate an immobilized polynucleotide that remains attached to the solid support. After the extension phase, the polynucleotides (e.g., polynucleotide template and its complementary product) may be denatured such that the template polynucleotide is released into solution and made available for hybridization to another immobilized primer. The polynucleotide template may be made available in 1, 2, 3, 4, 5 or more rounds of primer extension or may be washed out of the reaction after 1, 2, 3, 4, 5 or more rounds of primer extension.

In surface (or bridge) amplification, an immobilized polynucleotide template hybridizes to a surface oligonucleotide immobilized on a surface. The 3′ end of the immobilized polynucleotide template provides the template for a polymerase-catalyzed, template-directed elongation reaction (e.g., primer extension) extending from the immobilized surface oligonucleotide. The resulting double-stranded product “bridges” the two surface oligonucleotides and both strands are covalently attached to the support. In the next cycle, following denaturation that yields a pair of single strands (the immobilized polynucleotide template and the extended-primer product) immobilized to the surface, both immobilized strands can serve as templates for new primer extension. Examples of bridge amplification can be found in U.S. Pat. No. 7,790,418; U.S. Pat. No. 7,972,820; WO 2000/018957; U.S. Pat. No. 7,790,418; and Adessi et al., Nucleic Acids Research (2000): 28(20): E87).

In some embodiments, after bridge amplification and while the double stranded bridge complex exists, the surface may be treated with an exonuclease. The exonuclease will remove at least a portion of surface oligonucleotides that are not participating in a double stranded bridged structure. The exonuclease may completely remove individual surface oligonucleotides or remove portions of individual surface oligonucleotides. Treating the surface with an exonuclease prior to applying the sequencing methods of the present disclosure may result in a lower background signal during sequencing.

Any suitable exonuclease may be used. Examples of suitable exonucleases include Exonuclease I, Exonuclease T, and Exonuclease VII (all are available from New England Biolabs, MA). Preferably, the exonuclease has a high specificity for single stranded DNA over double stranded DNA.

Amplification may be used to produce colonies of immobilized polynucleotide templates. For example, the methods can produce clustered arrays of polynucleotide template colonies, analogous to those described in U.S. Pat. No. 7,115,400; U.S. Pat. No. 7,985,565; WO 00/18957; and WO 98/44151, which are incorporated by reference herein in their entireties. “Clusters” and “colonies” are used interchangeably and refer to a plurality of copies of a polynucleotide template having the same sequence and/or complements thereof attached to a surface. Typically, the cluster comprises a plurality of copies of a polynucleotide template having the same sequence and/or complements thereof, attached via their 5′ end to the surface. The copies of polynucleotide templates making up the clusters may be in a single or double stranded form.

The plurality of polynucleotide templates may be in a cluster, each cluster containing polynucleotide templates of the same sequence. A plurality of clusters can be sequenced, each cluster comprising polynucleotide templates of the same sequence. Optionally, the sequence of the polynucleotide templates in a first cluster is different from the sequence of the polynucleotide templates of a second cluster. Optionally, the cluster is formed by annealing a polynucleotide template to a primer on a surface and amplifying the polynucleotide template under conditions to form the cluster that includes the plurality of polynucleotide templates of the same sequence. Amplification can be thermal or isothermal.

Each colony may include a plurality of polynucleotide templates of the same sequences. In some embodiments, the sequence of the polynucleotide templates of one colony is different from the sequence of the polynucleotide templates of another colony. Thus, each colony comprises polynucleotide templates having different target nucleic acid sequences. All the immobilized polynucleotide templates in a colony are typically produced by amplification of the same polynucleotide template. In some embodiments, it is possible that a colony of immobilized polynucleotide templates includes one or more primers without an immobilized polynucleotide template to which another polynucleotide of different sequence may bind upon additional application of solutions containing free or unbound polynucleotide templates.

### Sequencing the Target Nucleic Acids

The present disclosure is directed to, among other things, methods for sequencing polynucleotide templates that contain one or more target nucleic acids. Particularly, the present disclosure is directed at the sequencing of polynucleotide templates using surface oligonucleotides as the sequencing primers (surface primers). The surface primers may comprise the amplification primers, or a portion thereof. Accordingly, the sequencing methods may be carried out on template polynucleotides that have been immobilized to a surface and amplified as described above.

Prior to sequencing, a strand of a double-stranded surface-bound polynucleotide may be cleaved in a process that results in the surface sequencing primer. The strand of the surface-bound polynucleotide may be cleaved in an adapter region of a template polynucleotide or may be cleaved in a region of the surface oligonucleotide (amplification primer) to which the template polynucleotide is bound.

In some embodiments, the sequencing comprises sequencing a single-stranded polynucleotide. In some embodiments, the double-stranded surface-bound polynucleotide may be denatured, and the cleaved strand may be washed away, leaving a single strand hybridized to the surface primer. Sequencing may occur using the surface primer and the remaining hybridized single strand.

In some embodiments, the sequencing comprises sequencing a strand of a double-stranded polynucleotide. For example, following cleavage and generation of the surface primer, sequencing may occur without removal of the cleaved strand. Sequencing of the strand of the double-stranded polynucleotide may proceed via strand displacement, nick translation, or any other suitable mechanism. The sequencing methods of the present disclosure preferably use sequencing by synthesis (SBS) to elucidate the nucleotide sequence of regions of interest on the polynucleotide templates. SBS techniques include, but are not limited to, the Genome Analyzer systems (Illumina Inc., San Diego, CA) and the True Single Molecule Sequencing (tSMS)™ systems (Helicos BioSciences Corporation, Cambridge, MA). In the SBS technique, a number of sequencing by synthesis reactions are used to elucidate the identity of a plurality of bases at target positions within a target sequence. In conventional SBS, these reactions rely on the use of a target nucleic acid sequence having at least two domains; a first domain to which a sequencing primer will hybridize; and an adjacent second domain, for which sequence information is desired. When SBS is used in conjunction with the sequencing methods of the current disclosure, a primer attached to the surface derived from the surface oligonucleotides (e.g., surface primer) is the sequencing primer and the second domain is the target nucleic acid sequence and/or other sequences of the template polynucleotide such as indexes. As will be described in detail below, at least a portion of the template polynucleotide template (e.g., at least a portion of the adapter) may be already hybridized to the surface primer. Because the surface primer is serving as the sequencing primer, no additional sequencing primer is needed. This may allow for a reduction in the number of sequencing reagents. With the reduction in the number of sequencing reagents, the methods of the present disclosure may be more economically and environmentally friendly.

After formation of an initial sequencing complex (a template strand hybridized to a surface primer) as described above, a chain extension enzyme may be used to add deoxynucleotide triphosphates (dNTPs) to the surface sequencing primer, and each addition of dNTPs may be read to determine the identity of the added dNTP. This may proceed for many cycles. The sequence for which the nucleotide identity is determined is generally termed a “read.” Read lengths may be greater than 5, greater than 10, greater than 20, greater than 50, greater than 100, greater than 200, greater than 300, or greater than 400 nucleotides in length.

In some SBS embodiments, the polynucleotide template is hybridized with a surface primer and incubated in the presence of a polymerase and one or more labeled nucleotides that include a 3′ blocking group. Examples of labeled nucleotides that include a blocking group can be found in WO 2004/018497. The surface primer is extended such that the labeled nucleotide is incorporated. The presence of the blocking group permits only one round of incorporation, that is, the incorporation of a single nucleotide. The presence of the label permits identification of the incorporated nucleotide. In some embodiments, the label is a fluorescent label. A plurality of homogenous single nucleotide bases can be added during each cycle, such as used in the True Single Molecule Sequencing (tSMS)™ systems (Helicos BioSciences Corporation, Cambridge, MA).Alternatively, all four nucleotide bases can be added during each cycle simultaneously, such as used in the Genome Analyzer systems (Illumina Inc., San Diego, CA), particularly when each base is associated with a distinguishable label. After identifying the incorporated nucleotide by its corresponding label, both the label and the blocking group can be removed, thereby allowing a subsequent round of incorporation and identification. Determining the identity of the added nucleotide base includes, in some embodiments, repeated exposure of the newly added labeled bases to a light source that can induce a detectable emission due the addition of a specific nucleotide. In some embodiments, the label is a fluorescent label.

In some embodiments, the nucleotides used in SBS do not include a label, for example when pyrosequencing is used. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. No. 6,210,891; U.S. Pat. No. 6,258,568 and U.S. Pat. No. 6,274,320). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Because the incorporation of any dNTP into a growing chain releases pyrophosphate, the four dNTP bases must be added to the system in separate steps. Useful fluidic systems, detectors, and procedures that can be used for application of pyrosequencing to arrays of the present disclosure are described, for example, in WO2012058096A1; U.S. Pat. Pub. No. 2005/0191698 A1; U.S. Pat. No. 7,595,883; and U.S. Pat. No. 7,244,559.

Sequencing-by-ligation SBS reactions such as those described in Shendure et al. Science 309:1728-1732 (2005); U.S. Pat. No. 5,599,675; and U.S. Pat. No. 5,750,341 may also be used. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977. In both sequencing-by-ligation and sequencing-by-hybridization procedures, template nucleic acids (e.g., a target nucleic acid or amplicons thereof) that are present at sites of an array are subjected to repeated cycles of oligonucleotide delivery and detection. Fluidic systems for SBS methods can be readily adapted for delivery of reagents for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some embodiments can use methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in U.S. Pat. No. 8,262,900; U.S. Pat. No. 7,948,015; U.S. Pat. Pub. 2010/0137143 A1; or U.S. Pat. No. 8,349,167.

The sequencing methods disclosed herein are particularly useful when used in conjunction with SBS. In addition, the sequencing methods described herein may be particularly useful for sequencing from an array clusters of polynucleotide templates, where multiple sequences can be read simultaneously from multiple clusters on the array since each nucleotide at each position can be identified based on its identifiable label. Exemplary methods are described in U.S. Pat. No. 7,754,429; U.S. Pat. No. 7,785,796; and U.S. Pat. No. 7,771,973, each of which is incorporated herein by reference.

In some embodiments, where the polynucleotide templates include one or more index sequences, the index sequences may be sequenced using SBS.

In some embodiments, SBS involves several rounds of incorporation of nucleotides for which the identity of the incorporated nucleotides are not determined. Such rounds of incorporation may be referred to as “dark cycles.” Dark cycling involves the sequential incorporation of nucleotides containing a 5′ blocking group and subsequent blocking group removal. Dark cycles may be used to skip the reading of index sequences, universal sequences, and/or any other sequence where the identity is not desired to be determined. Each cycle of a dark cycle includes the incorporation of a nucleotide. Any suitable number of dark cycles of incorporation may be performed to effectively reach the portion of the polynucleotide template where determining the nucleotide sequence is desired. For example, 2 to 150 dark incorporation cycles may be performed, such as 3 to 100, 5 to 50, or 6 to 25 dark cycles. The sequence of the polynucleotide template strand to which the extended surface primer is complementary during the dark cycles is preferably known. Once the appropriate number of dark cycles of incorporation are performed, SBS (determining the identity of the nucleotides incorporated in subsequent cycles) may be performed.

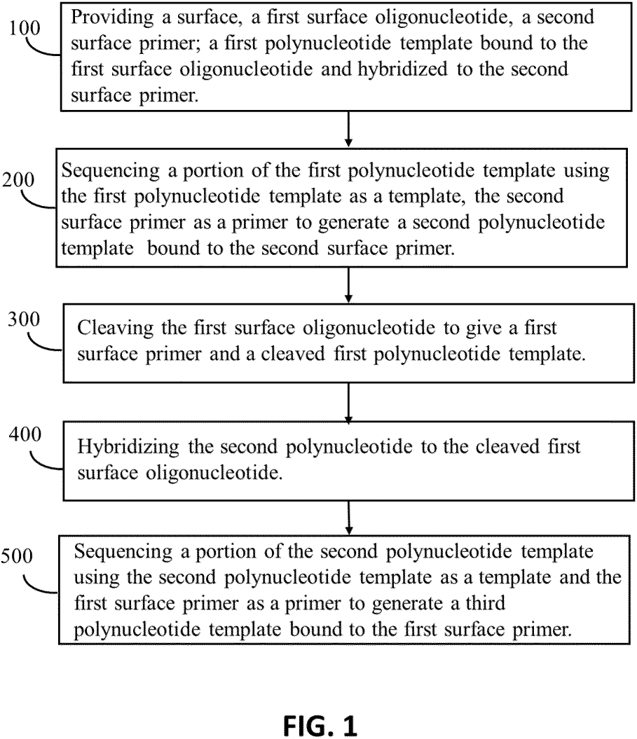

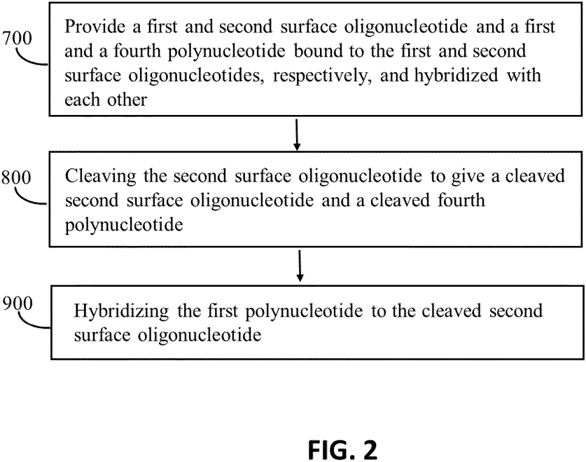

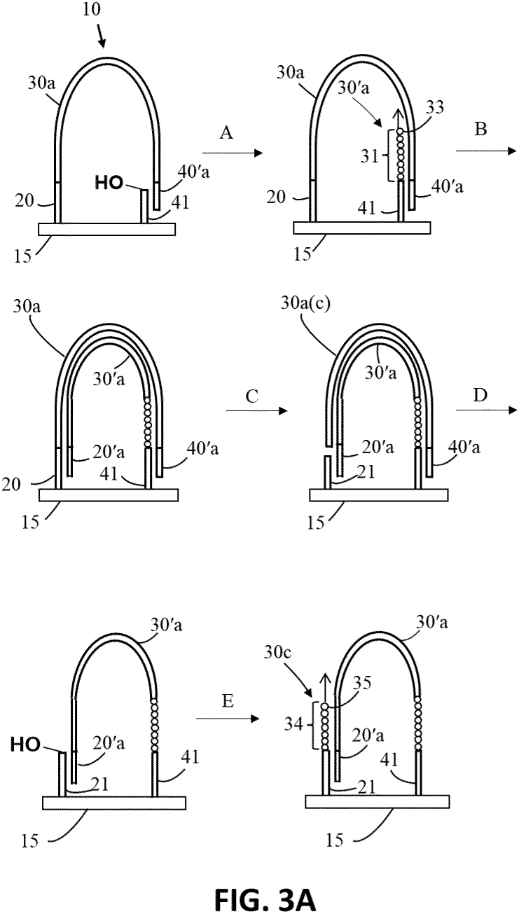

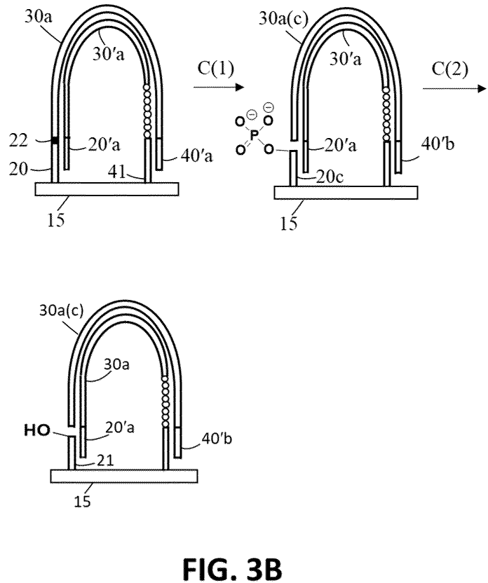

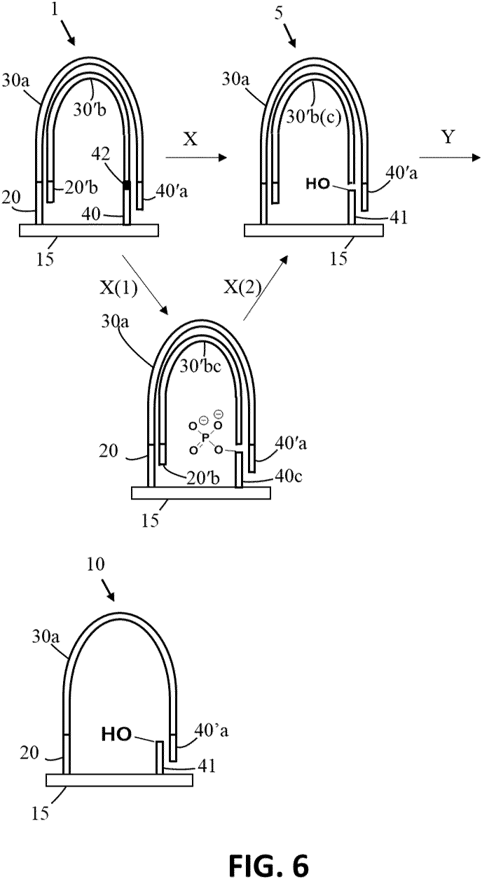

FIGS. 1, 2, 3A-B, 4, 5, and 6 are referenced to illustrate embodiments consistent with the present disclosure. FIG. 1 and FIGS. 3A-B, 4, and 5 refer to the various sequencing methods of the present disclosure. FIG. 2 and FIG. 6 refer to pre-sequencing methods of the present disclosure. For clarity, the description of each element and step in the figures is described in the singular. However, it should be understood that the sequencing and pre-sequencing methods described herein may be applied to arrays or cluster of polynucleotides provided as describe previously in order to accomplish massive parallel sequencing.

FIG. 1 is a flow chart illustrating an overview of the sequencing methods consistent with some embodiments of the present disclosure. The method includes providing a surface, a first surface oligonucleotide bound to the surface at its 5′ end, a second surface primer bound to the surface at its 5′ end and having a free 3′ end, and a first polynucleotide template covalently bound to the first surface oligonucleotide its 5′ end and at least of portion in proximity to its (the first polynucleotide template) free 3′ end hybridized to at least a portion of the second surface primer (100). The method further includes sequencing at least a portion of the first polynucleotide template by extending the second surface primer from its free 3′ end using the first polynucleotide template and the second surface primer as a primer to generate a second polynucleotide template that is covalently bound to the second surface primer and has a free 3′ end (200). The second polynucleotide template includes at least a portion that is complementary to the first polynucleotide template including a first read region and at least a portion in proximity to the free 3′ end that is complementary to at least a portion of the first surface oligonucleotide. The method further includes cleaving the first surface oligonucleotide to produce a first surface primer that is bound to the surface its 5′ end and has a free 3′, and a cleaved first polynucleotide template that has a free 5′ end and free 3′ end (300). The method may optionally include hybridizing at least a portion of the second polynucleotide template in proximity to its free 3′ end to the first surface primer (400), if the portion of the second polynucleotide template in proximity to its free 3′ end is not already hybridized to the first surface primer. The method further includes sequencing at least a portion of the second polynucleotide template by extending the first surface primer from its free 3′ end using the second polynucleotide template as a template and at least a portion of the first surface primer as a primer to generate a third polynucleotide template (500). The third polynucleotide template includes at least a portion that is complementary to the second polynucleotide template that includes a second read region.

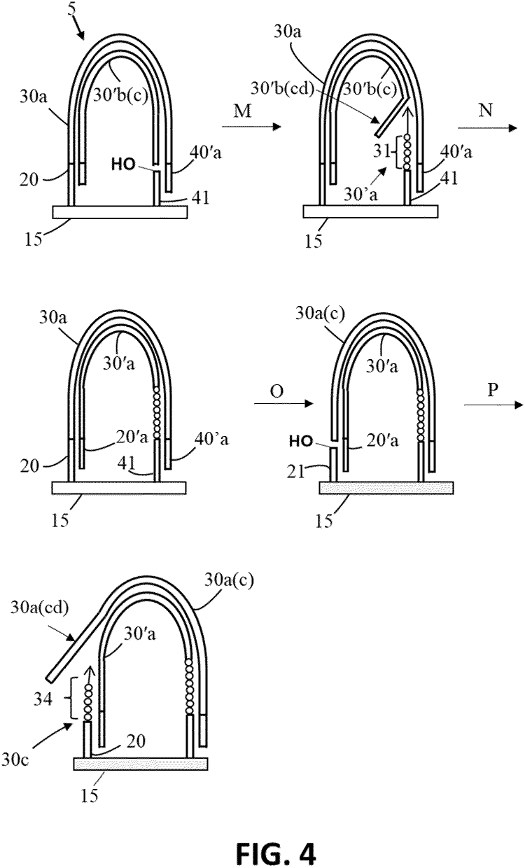

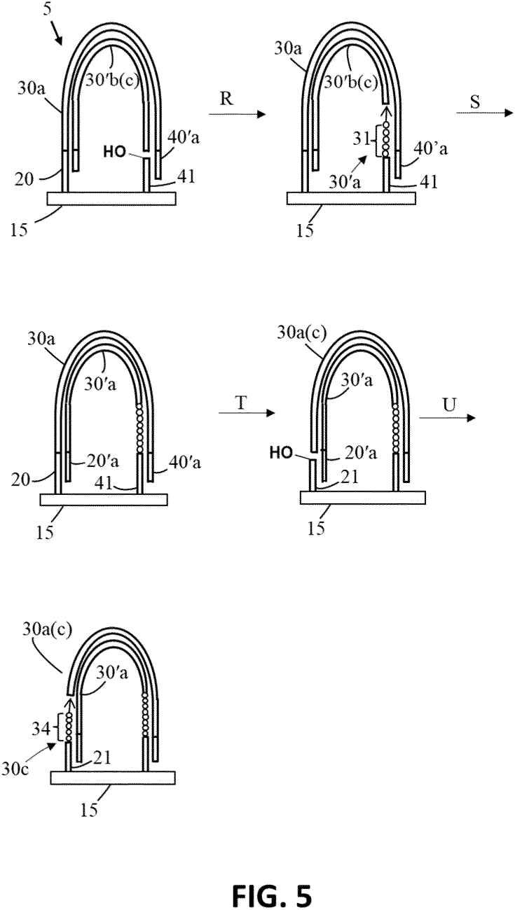

For purposes of illustration, aspects of the sequencing methods consistent with embodiments of the present disclosure are describe below with reference to FIGS. 3A-B, 4, and 5. FIGS. 3A-3B illustrate a single stranded surface sequencing method consistent with some embodiments of the present disclosure. FIG. 4 illustrates a double stranded surface sequencing via displacement method consistent with some embodiments of the present disclosure. FIG. 5 illustrates a double stranded surface sequencing via nick translation method consistent with some embodiments the present disclosure.

### Single Strand Surface Sequencing (ssSurfSeq)

FIGS. 3A-B provide a schematic overview of a single strand surface sequencing method consistent with some embodiments of the present disclosure. The workflow includes providing a pre-sequencing complex 10. Pre-sequencing complex 10 includes a surface 15, a first surface oligonucleotide 20, a second surface primer 41, and a first polynucleotide template 30a. The first surface oligonucleotide 20 is bound to the surface 15 at its 5′ end. The second surface primer 41 is bound to the surface at its 5′ end and has a free 3′ end with a terminal hydroxyl at the 3′ position on the deoxyribose. The first polynucleotide template 30a is covalently bound to the first surface oligonucleotide 20 at its 5′ end and has a free 3′ end. The first polynucleotide template 30a includes a 3′ region 40′a that is hybridized to at least a portion of the second surface primer 41 to form a single stranded bridge structure. In some embodiments, the 3′ region 40′a includes at least a portion of an adapter previously ligated to the first polynucleotide template 30a. For example, the second surface primer 41 may include the P5 sequence and the 3′ region 40′a may include a P5′ sequence that is configured to hybridize to a P5 sequence.

In some embodiments the second surface primer 41 comprises at least a portion of a second surface oligonucleotide 40 (see, e.g., FIG. 6) that was immobilized on the surface. The second surface oligonucleotide 40 may be cleaved to give the second surface primer 41.

In some embodiments, pre-sequencing complex 10 is provided as described in reference to FIG. 6 later herein.

In step A of FIG. 3A, at least a portion of the first polynucleotide template 30a is sequenced as a first read region 31. Sequencing may include sequencing by synthesis where the second surface primer 41 is enzymatically extended in the 5′ to 3′ direction thereby creating a portion of a second polynucleotide template 30′a that is complementary to the first polynucleotide template 30a. The portion of the second polynucleotide template 30′a generated during sequencing is the first read region 31. The enzymatic extension uses the first polynucleotide template 30a as the template and at least a portion of the second surface primer 41 as the sequencing primer.

In step B of FIG. 3A, the second polynucleotide template 30′a is extended from the first read region 31 via the incorporation of nucleotides and the use of the first polynucleotide template 30a as the template. The nucleotides incorporated may be blocked nucleotides, such as those used in SBS, or unblock nucleotides allowing for rapid chain extension. The second polynucleotide template 30′a is covalently bonded to the second surface primer 41 its 5′ end and has a free 3′ end. The second polynucleotide template 30′a is complementary to the first polynucleotide template 30a and includes the first read region 31 proximate to its 5′ end and a 3′ end region 20′a that is complementary to at least a portion of the first surface oligonucleotide 20. The first polynucleotide template 30a and the second polynucleotide template 30′a are hybridized in a double stranded bridged structure.

Steps C and D of FIG. 3A, illustrate the process of linearization. As referred to herein, “linearization” is the selective removal of a specific strand of DNA, such as a polynucleotide template. Examples of suitable methods for linearization are described herein and are described in more detail in WO 2007/010251 and U.S. Pat. No. 8,431,348. Linearization includes two general steps. First, one strand of a double stranded DNA, such as a double stranded bridged structure (30a in step C), is cleaved (step C) to produce a cleaved strand (30a(c) in step C). Second, the cleaved strand that no longer has any covalent attachment to the surface is removed (30a(c) in step D).

In step C of the linearization process, the first surface oligonucleotide 20 is cleaved to produce a first surface primer 21 and a cleaved first polynucleotide template 30a(c). The first surface primer 21 has a free 3′ end that includes a terminal hydroxyl at the 3′ position on the deoxyribose. Various cleavage methods may be used including, for example, abasic cleavage, chemical cleavage, cleavage of ribonucleotides, photochemical cleavage, hemimethylated DNA cleavage, nicking endonuclease cleavage, and restriction enzyme cleavage, some of which are described in more detail below.

### Abasic Cleavage

In some embodiments, abasic cleavage is used to cleave the first surface oligonucleotide 20 as illustrated in FIG. 3B. In some embodiments, the first surface oligonucleotide 20 includes a first excisable base 22. The first excisable base 22 is generally configured to be removed from the first surface oligonucleotide 20. The first excisable base 22 may be located anywhere along the first surface oligonucleotide 20.

In some embodiments (not shown) the excisable base may be located anywhere on the polynucleotide template, for example, in the 5′ adapter region.

In some embodiments, the first excisable base 22 is removed from the first surface oligonucleotide 20 resulting in an abasic site. An “abasic site” is a nucleotide position in a polynucleotide from which the base component has been removed. Abasic sites can be formed chemically under artificial conditions or by the action of enzymes.

In some embodiments, an abasic site may be created at a pre-determined position on the first surface oligonucleotide 20. This can be achieved, by incorporating a specific excisable base at the pre-determined position. For example, the first excisable base 22 may be incorporated at a specific location in the first surface oligonucleotide 20.

The first excisable base 22 may be any base or modified base that can be removed from a double stranded DNA. Example excisable bases include, but are not limited to, deoxyuridine (dU); 8-oxo-guanine (8-oxo-G); deoxyinosine; 7,8-dihydro-8-oxoguanine (8-oxoguanine); 8-oxoadenine; fapy-guanine; methyl-fapy-guanine fapy-adenine; aflatoxin B1-fapy-guanine; 5-hydroxy-cytosine; 5-hydroxy-uracil; and the like. In some embodiments, deoxyuridine may be provided by heat assisted deamination of 5-methyl cytosine (methyl-C), bisulfite assisted deamination of methyl-C, or both. Enzymes that may be used to create an abasic site include, but are not limited to, uracil DNA glycosylase (UDG); a uracil specific excision reagent enzyme such as USER (available from New England BioLabs located in Ipswich, MA); FPG glycosylase; AlkA glycosylase; oxoguanine glycosylase, and the like. In some embodiments, the first excisable base 22 is deoxyuridine (dU). In some such embodiments, UDG and/or an uracil specific excision reagent enzyme is used to create the abasic site. In some embodiments, the first excisable base 22 is 8-oxo-G. In some such embodiments , FPG glycosylase is used to the create an abasic site.

Once formed, an abasic site may be cleaved providing a means for site-specific cleavage of polynucleotide, such as a polynucleotide template. For example, removal of the abasic site generated after the removal of the first excisable base 22, will generate the cleaved first polynucleotide template 30a(c) that is no longer covalently attached to the surface 15. The polynucleotide strand that includes the abasic site can then be cleaved at the abasic site by treatment with endonuclease such as DNA glycosylase-lyase Endonuclease VIII, AP lyase, FPG glycosylase, heat, or alkali conditions to yield a 3′ phosphate on 3′ terminal end of the oligonucleotide that is attached to the surface (the first surface oligonucleotide 20 in FIG. 3B). In some embodiments, a mixture containing the appropriate glycosylase and one or more suitable endonucleases, typically in an activity ratio of at least about 2:1, is used to generate the abasic site and cleave the polynucleotide strand at the abasic site in a single step. For example, in some embodiments, the surface is treated with a mixture of uracil DNA glycosylase and endonuclease VII to generate an abasic site at the first excisable base 22 and cleave the polynucleotide strand at the abasic site generating a cleaved first polynucleotide template 30a(c) and a terminal 3′ phosphate on the free 3′ end of the first surface oligonucleotide 20.

In step C(2) of FIG. 3B, a hydroxyl at the 3′ position of the deoxyribose of free 3′ end of the cleaved first surface oligonucleotide 20c is generated to give a first surface primer 21. Examples of enzymes that can be used to generate a hydroxyl from a phosphate include, but are not limited to, T4 polynucleotide kinase (T4PNK), Endonuclease IV, and suitable phosphatases such as calf intestinal phosphatase, shrimp alkaline phosphatase, and pyrococcus abysii alkaline phosphatase.

Advantages of the abasic cleavage method may include the option of releasing a free 3′ phosphate group on the cleaved strand, which after treatment to generate terminal 3′ hydroxyl group can provide an initiation point for sequencing. Because the cleavage reaction requires a residue, e.g., deoxyuridine, which does not occur naturally in DNA, but is otherwise independent of sequence context, if only one non-natural base is included there is no possibility of glycosylase-mediated cleavage occurring elsewhere at unwanted positions in the double stranded DNA bridged structure. An advantage gained by cleavage of abasic sites in a double-stranded section of an immobilized polynucleotide templates generated by action of UDG on uracil is that the first base incorporated in a sequencing-by-synthesis reaction initiating at the free 3′ hydroxyl group formed by cleavage will always be T. As a result, for all clonal clusters at different amplification sites of an array which are cleaved in this manner to produce sequencing templates the first base universally incorporated across the whole array will be T. This can provide a sequence-independent assay for individual cluster intensity at the start of a sequencing run.

In some embodiments, the abasic cleavage of the first excisable base (step C(1)) and the generation of the hydroxyl at the 3′ position of the deoxyribose of the free 3′ end of the first surface oligonucleotide 20 (step C(2)) may be accomplished in one step C (FIG. 3A). In some embodiments, a single reagent may be used to excise the first excisable base 22 and generate a terminal hydroxyl at the 3′ position on the deoxyribose of the free 3′ end of the first surface oligonucleotide 20 to give a first surface primer 21. For example, EndoQ from Pyrococcus furious (Pfu) recognizes and cuts the 5′ phosphodiester bond of uracil to generate a hydroxyl at the 3′ position of the deoxyribose on the nucleotide that is on the 5′ side of uracil (Ishino et al., Sci Rep. 2016 May 6;6:25532. Doi: 10.1038/srep25532; Ishino et al., Nucleic Acids Res. 2015 Mar 11;43(5):2853-63. Doi: 10.1093/nar/gkv121). Thus, EndoQ may be used to both cleave the first excisable base 22 and generate a hydroxyl at the 3′ position of the deoxyribose of the free 3′ end of the first surface oligonucleotide 20.

Preferably, in some embodiments, the steps of C or C(1) and C(2) may occur while the first polynucleotide template 30a is hybridized to the second polynucleotide template 30′a in a double stranded bridged structure.

### Chemical Cleavage

In some embodiments, chemical cleavage methods are used to cleave the first surface oligonucleotide 20. The term “chemical cleavage” encompasses any method which uses a non-enzymatic chemical reagent in order to promote/achieve cleavage of the original single-stranded polynucleotide template. If required, the single-stranded amplicon may include one or more non-nucleotide chemical moieties and/or non-natural nucleotides and/or non-natural backbone linkages, such as allyl-dNTPs, in order to permit a chemical cleavage reaction.

In some embodiments, the surface oligonucleotides and/or template polynucleotides includes one or more ally-dNTPs such as, for example, allyl-T, allyl-A, allyl-G, or allyl-C. The allyl-dNTP provides a site for chemical cleavage. In some embodiments, the allyl-dNTP allows for single step or two step cleavage and 3′ hydroxyl generation, e.g., as shown in FIG. 3A or in FIG. 3B.

In some embodiments, a surface oligonucleotide and/or a template polynucleotide comprising an allyl-dNTP is cleaved and hydroxylated in two steps by treatment with Pd(0) and a hydroxyl forming reagent. In some embodiments, the first step (e.g., step C1 in FIG. 3B) includes cleavage with Pd(0) to produce a cleaved first surface oligonucleotide 20c that has a free 3′ end that includes a terminal phosphate at the 3′ carbon of the deoxyribose sugar. In such embodiments, further treatment with one or more hydroxyl forming groups converts the phosphate into a terminal hydroxyl (e.g., step C2 in FIG. 3B) thereby generating the first surface primer 21. Examples of hydroxyl forming groups include, but are not limited to, T4 polynucleotide kinase (T4PNK), endonuclease IV, suitable phosphatases such as those described herein, or combinations thereof.

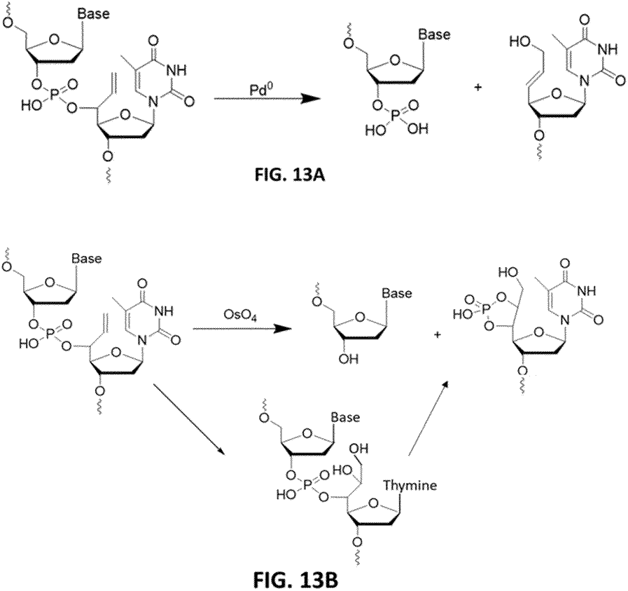

FIG. 13A shows an example cleavage reaction at the allyl-T of an oligonucleotide using Pd(0). In FIG. 13A, the oligonucleotide (e.g., the first surface oligonucleotide 20 or the first polynucleotide template 30a of FIGS. 3A-B) includes an allyl-T. Treatment with Pd(0) results in the cleavage of the oligonucleotide strand at the 5′ carbon of the deoxyribose sugar of allyl-T to produce two oligonucleotide strands. The 3′ end of the first oligonucleotide strand (e.g., the cleaved first surface oligonucleotide 20c of FIGS. 3A-B) has a phosphate group at the 3′ carbon of the terminal deoxyribose sugar. The 5′ end of the second oligonucleotide strand (e.g., the cleaved first polynucleotide template 30a(c) of FIGS. 3A-B) has an alkene and an alcohol extending from the 5′ carbon of the deoxyribose sugar of the allyl-T. The terminal phosphate at the 3′ carbon of the deoxyribose sugar of the first oligonucleotide (e.g., the cleaved first surface oligonucleotide 20c of FIGS. 3A-B) can be further converted to a terminal hydroxyl through treatment with one or more hydroxyl forming reagents such as, for example, T4 polynucleotide kinase (T4PNK), endonuclease IV, or combinations thereof.

In some embodiments, a surface oligonucleotide and/or a template polynucleotide comprising an allyl-dNTP is cleaved to produce a 3′ hydroxyl in a single step (e.g., step C in FIG. 3A) via treatment with reagent or reagents that dihydroxylate the alkene of the allyl-dNTP. In such embodiments, cleavage of the first surface oligonucleotide 20 to form a first surface primer 21 that has a free 3′ end that includes a terminal hydroxyl at the 3′ carbon of the deoxyribose includes treatment with one or more reagents that allow for a dihydroxylation reaction.

Dihydroxylation is the formation of a vicinal diol from an alkene. Without wishing to be bound by theory, it is thought that when an oligonucleotide containing an allyl-dNTP is subjected to a dihydroxylation reagent or reagents, the vicinal diol intermediate will decompose to form two oligonucleotides: a first oligonucleotide that includes a free 3′ end having a terminal hydroxyl on the 3′carbon of the terminal deoxyribose and a second oligonucleotide.

Any suitable dihydroxylation reagent or mixture of reagents may be used. Various alkene dihydroxylation reactions and the corresponding reagents are known, such as, for example, Sharpless asymmetric dihydroxylation, Milas dihydroxylation, Upjohn dihydroxylation, and Prevost and Woodward dihydroxylation. The Sharpless asymmetric dihydroxylation, Milas dihydroxylation, and Upjohn dihydroxylation use a catalyst and a stoichiometric oxidant to accomplish the dihydroxylation reaction. A common catalyst is osmium tetroxide (OsO₄). Stoichiometric oxidants include, but are not limited to, K₃[Fe(CN)₆], peroxide, water, and N-methylmorpholine N-oxide (NMO). The Prevost and Woodward dihydroxylation use iodine (I₂) and a silver salt (e.g., OHCO₂Ag) to accomplish dihydroxylation.

In some embodiments, cleavage of the first surface oligonucleotide 20 to form a first surface primer 21 that has a free 3′ end that includes a terminal hydroxyl at the 3′ carbon of the deoxyribose, includes treatment with a catalyst and a stoichiometric oxidant. In some embodiments, the catalyst is osmium tetroxide. In embodiments, the stoichiometric oxidant is K₃[Fe(CN)₆], peroxide, N-methylmorpholine N-oxide (NMO), water, or any combination thereof. In embodiments, cleavage of the first surface oligonucleotide 20 to form a first surface primer 21 that has a free 3′ end that includes a terminal hydroxyl at the 3′ carbon of the deoxyribose, includes treatment with iodine and a silver salt.

In some embodiments, additional compounds, buffering agents, and/or solvents may be included in a dihydroxylation reaction. For example, various solvents may be included such as, water, t-butanol, isopropanol, or combinations thereof may be included in a dihydroxylation reaction.

In some embodiments in which OsO₄ is used, the OsO₄ may be formed in situ.

FIG. 13B shows an example cleavage reaction at the allyl-T of an oligonucleotide using a dihydroxylation reaction. In FIG. 13B the oligonucleotide (e.g., the first surface oligonucleotide 20 or the first polynucleotide template 30a of FIGS. 3A-B) includes an allyl-T. Treatment with the dihydroxylation reagent, osmium tetroxide (OsO₄) and a stoichiometric oxidant results in the cleavage of the oligonucleotide strand at the 5′ carbon of the deoxyribose sugar of allyl-T to produce two oligonucleotide strands. The 3′ end of the first oligonucleotide strand (e.g., the first surface primer 21 of FIGS. 3A-B) has a terminal hydroxyl group on 3′ carbon of the terminal deoxyribose sugar. Although the structure is unknown and not wishing to be bound by theory, the 5′ end of the second oligonucleotide strand (e.g., the cleaved first polynucleotide template 30a(c) of FIGS. 3A-B) is thought to have a five-member phosphate containing ring structure extending from the 5′ carbon of the terminal deoxyribose sugar of the allyl-T. The proposed five-member phosphate containing ring structure may decompose to form a different chemical group.

FIG. 13B also shows a proposed vicinal diol oligonucleotide intermediate structure that may occur post dihydroxylation but prior to separation of the oligonucleotide. Not wishing to be bound by theory, it is thought that the vicinal diol intermediate decomposes to give the first oligonucleotide and the second oligonucleotide having the terminal chemical groups as described above. In one embodiment, the surface oligonucleotides or polynucleotides includes a diol linkage which permits cleavage by treatment with periodate (e.g., sodium periodate). It will be appreciated that more than one diol can be included at the cleavage site. Diol linker units based on phosphoramidite chemistry suitable for incorporation into a surface oligonucleotides or polynucleotides are commercially available from Fidelity systems Inc. (Gaithersburg, MD., USA). One or more diol units may be incorporated into a surface oligonucleotides or polynucleotides using standard methods for automated chemical DNA synthesis. Hence, the surface oligonucleotides including one or more diol linkers can be conveniently prepared by chemical synthesis.

The diol linker is cleaved by treatment with a “cleaving agent,” which can be any substance that promotes cleavage of the diol. The preferred cleaving agent is periodate, such as aqueous sodium periodate (NaIO₄). Following treatment with the cleaving agent (e.g., periodate) to cleave the diol, the cleaved product may be treated with a “capping agent” in order to neutralize reactive species generated in the cleavage reaction. Suitable capping agents for this purpose include amines, such as ethanolamine. Advantageously, the capping agent (e.g., ethanolamine) can be included in a mixture with the cleaving agent (e.g., periodate) so that reactive species are capped as soon as they are formed. The resulting surface oligonucleotide may be treated to contain a 3′ hydroxyl group to enable use of the surface oligonucleotide as a primer for sequencing, chain extension, or sequencing and chain extension.

In another embodiment, the surface oligonucleotides or polynucleotides can include a disulfide group which permits cleavage with a chemical reducing agent, e.g., tris(2-carboxyethyl)-phosphate hydrochloride (TCEP).

After chemical cleavage, one or more additional reagents, such as a phosphatase, may be needed to generate a terminal 3′ hydroxyl on the surface oligonucleotide resulting in a surface primer.

### Cleavage of Ribonucleotides

Incorporation of one or more ribonucleotides into a polynucleotide, such as a surface oligonucleotide or a polynucleotide template, which is otherwise made up of deoxyribonucleotides (with or without additional non-nucleotide chemical moieties, non-natural bases or non-natural backbone linkages) can provide a site for cleavage using a chemical agent capable of selectively cleaving the phosphodiester bond between a deoxyribonucleotide and a ribonucleotide or using a ribonuclease (RNAse). The surface oligonucleotide (e.g., the first surface oligonucleotide 20 of FIGS. 3A-B) may be cleaved at a site containing one or more consecutive ribonucleotides using such a chemical cleavage agent or an RNase. In one embodiment, the strand to be cleaved contains a single ribonucleotide to provide a site for chemical cleavage.

Suitable chemical cleavage agents capable of selectively cleaving the phosphodiester bond between a deoxyribonucleotide and a ribonucleotide include metal ions, for example rare-earth metal ions (e.g., La³⁺, Tm³⁺, Yb³⁺, or Lu³⁺;Chen et al. Biotechniques. 2002, 32: 518-520; Komiyama et al. Chem. Commun. 1999, 1443-1451)), Fe(III) or Cu(III), or exposure to elevated pH (e.g., treatment with a base such as sodium hydroxide). By “selective cleavage of the phosphodiester bond between a deoxyribonucleotide and a ribonucleotide” is meant that the chemical cleavage agent is not capable of cleaving the phosphodiester bond between two deoxyribonucleotides under the same conditions.

The base composition of the ribonucleotide(s) is generally not material but can be selected in order to optimize chemical (or enzymatic) cleavage. By way of example, rUMP or rCMP are generally preferred if cleavage is to be carried out by exposure to metal ions, especially rare earth metal ions.

The phosphodiester bond between a ribonucleotide and a deoxyribonucleotide, or between two ribonucleotides may also be cleaved by an RNase. Any endocytic ribonuclease of appropriate substrate specificity can be used for this purpose. For cleavage with a ribonuclease it is preferred to include two or more consecutive ribonucleotides, such as from 2 to 10 or from 5 to 10 consecutive ribonucleotides. The precise sequence of the ribonucleotides is generally not material, except that certain RNases have specificity for cleavage after certain residues. Suitable RNases include, for example, RNaseA, which cleaves after C and U residues. Hence, when cleaving with RNaseA the cleavage site must include at least one ribonucleotide which is C or U.

Surface oligonucleotides or polynucleotide templates incorporating one or more ribonucleotides can be readily synthesized using standard techniques for oligonucleotide chemical synthesis with appropriate ribonucleotide precursors.

After ribonuclease cleavage, one or more additional reagents, such as a phosphatase, may be needed to generate a terminal 3′ hydroxyl on the surface oligonucleotide resulting in a surface primer.

### Photochemical Cleavage

The term “photochemical cleavage” encompasses any method which uses light energy in order to achieve cleavage of a nucleic acid. A site for photochemical cleavage can be provided by a non-nucleotide chemical spacer unit in the surface oligonucleotide and/or the polynucleotide templates. Suitable photochemical cleavable spacers include the PC spacer phosphoramidite (4-(4,4′-Dimethoxytrityloxy)butyramidomethyl)-1-(2-nitrophenyl)-ethyl]-2-cyanoethyl-(N,N-diisopropyl)-phosphoramidite) supplied by Glen Research, Sterling, Va., USA (cat number 10-4913-XX) which has the structure:

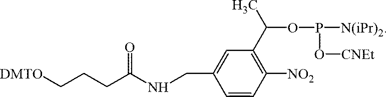

The spacer unit can be cleaved by exposure to a UV light source.

This spacer unit can be attached to the 5′ end of a polynucleotide, together with a thiophosphate group which permits attachment to a solid surface using standard techniques for chemical synthesis of oligonucleotides.

After photochemical cleavage, one or more additional reagents, such as a phosphatase, may be needed to generate a terminal 3′ hydroxyl on the surface oligonucleotide resulting in a surface primer.

### Cleavage of Hemimethylated DNA

Site-specific cleavage of the surface oligonucleotide can also be achieved by incorporating one or more methylated nucleotides into the surface oligonucleotide and/or the polynucleotide template, and then cleaving with an endonuclease enzyme specific for a recognition sequence including the methylated nucleotide(s).

The methylated nucleotide(s) will be opposite of non-methylated deoxyribonucleotides on the complementary strand, such that annealing of the two strands produces a hemimethylated duplex structure. The hemimethylated duplex may then be cleaved by the action of a suitable endonuclease.

Surface oligonucleotides and/or polynucleotide templates incorporating one or more methylated nucleotides may be prepared using standard techniques for automated DNA synthesis, using appropriately methylated nucleotide precursors.

After cleavage of hemimethylated DNA, one or more additional reagents, such as a phosphatase, may be needed to generate a terminal 3′ hydroxyl on the surface oligonucleotide resulting in a surface primer.

### Nicking Endonuclease Cleavage

Nicking endonucleases are enzymes that selectively cleave or “nick” one strand of a double-stranded nucleic acid. Essentially any nicking endonuclease may be used, provided that a suitable recognition sequence can be included at the cleavage site present on the nucleic acid. Examples of nicking endonucleases include, but are not limited to, Nt.BspQI, Nt.CviPII, Nt.Btsl, and Nb.Bsml (all available from New England Biolabs, MA). Preferably, endonucleases that have long recognition sequences (e.g., 12-40 bp), such as homing endonucleases, are used as nicking endonuclease in order to prevent nonspecific nicking of the polynucleotide template. Homing endonucleases may be converted to nicking endonucleases for example, as described in Niu et al, (2008) JMB Vol 382: 188-20 and Molina et al., (2015) JBC Vol 290: 18534 - 18544. Examples of commercially available homing endonucleases that are nicking endonucleases include, but are not limited to, I-CeuI, I-SceI, PI-PspI, and PI-SceI (all available from New England Biolabs, MA).

After nicking endonuclease cleavage, one or more additional reagents, such as a phosphatase, may be needed to generate a terminal 3′ hydroxyl on the surface oligonucleotide resulting in a surface primer.

Referring again to FIG. 3A, in step D, the cleaved first polynucleotide template 30a(c) is removed. Cleavage of the first surface oligonucleotide 20 results in a cleaved first polynucleotide 30a(c) that has newly formed free 5′ end and a free 3′, thus the cleaved first polynucleotide is 30a(c) not covalently attached to the surface 15. For example, as shown in FIG. 3A, the second polynucleotide template 30′a of the bridged structure remains covalently bound at its 5′ end to the second surface primer 41 but the first polynucleotide template 30a is no longer covalently bound to the first surface oligonucleotide 20 due to the cleavage (shown as 30a(c)). The cleaved first polynucleotide template 30a(c) can be completely removed from the surface by exposing the surface to suitable conditions. In some embodiments, removal includes denaturation.

Denaturing may be accomplished through one or more of thermal, chemical, and enzymatic means. For example, the surface may be heated to a temperature greater than the melting point of the first polynucleotide template 30a. Chemical denaturation may be accomplished through exposing the DNA to solvents such as dimethyl sulfoxide, dimethylformamide, isopropanol, ethanol, formamide, or propylene glycol; and salts such as guanidine, sodium salicylate, urea, or sodium chloride; or any combination thereof.

In some embodiments, removal is accomplished enzymatically by an exonuclease. In one embodiment, an exonuclease is a 5′-3′ DNA exonuclease. Optionally, the 5′-3′ DNA exonuclease has a bias for double stranded DNA. Examples of such exonucleases include, but are not limited to, T7 exonuclease and Exonuclease III (available from New England Biolabs). Optionally, the 5′-3′ DNA exonuclease has a bias for double stranded DNA having a 5′ phosphate at the 5′ end. An example of such an exonuclease is lambda exonuclease (available from New England Biolabs).

In addition to the final step of linearization, the removal of the polynucleotide that is no longer immobilized, in some embodiments, step D further includes hybridizing the free 3′ end region 20′a of the second polynucleotide template 30′a to at least a portion of the first surface primer 21 to give a single strand bridge structure (FIG. 3A). Hybridization may be accomplished by cooling the polynucleotides below their thermal melting temperatures. If the free 3′ end region 20′a of the second polynucleotide template 30′a remains hybridized to at least a portion of the first surface primer 21 during the linearization process, a separate hybridization step may not be needed.

In step E of FIG. 3A, at least a portion of the second polynucleotide template 30′a is sequenced as a second read region 34. Sequencing may include sequencing by synthesis where the second surface primer 21 is enzymatically extended in the 5′ to 3′ direction thereby creating a portion of a third polynucleotide template 30c that is complementary to the second polynucleotide template 30′a. The portion of the third polynucleotide template 30c generated in the extension is the second read region 34. The enzymatic extension uses the second polynucleotide template 30′a as the template and at least a portion of the first surface primer 21 as the sequencing primer.

In some embodiments, it may be desirable to sequence a polynucleotide template while the polynucleotide template is hybridized to another polynucleotide template in a double stranded structure, otherwise termed “double stranded sequencing” herein. Double stranded sequencing methods may decrease the likelihood of the formation of secondary structures such as G-quadraplexes that may form when the polynucleotide template is in single strand form. As such, double stranded sequencing methods may advantageously allow for higher sequencing accuracy relative to single stranded sequencing methods when single stranded nucleotide sequences form secondary structures that may be detrimental to sequencing.

### Double Stranded Surface Sequencing Via Strand Displacement (dsSurfSeq(Displacement))

FIG. 4 is a schematic overview of a double stranded surface sequencing via strand displacement method consistent with the embodiments of the present disclosure. The workflow includes providing a pre-sequencing complex 5. Similar to the single strand surface sequencing methods of the present disclosure, the pre-sequencing complex 5 includes a surface 15, a first surface oligonucleotide 20, a second surface primer 41, and a first polynucleotide template 30a. The first surface oligonucleotide 20 is bound to the surface 15 at its 5′ end. The second surface primer 41 is bound to the surface at its 5′ end and has a free 3′ end with a terminal hydroxyl at the 3′ position on the deoxyribose. The first polynucleotide template 30a is covalently bound to the first surface oligonucleotide 20 at its 5′ end. The first polynucleotide template includes a 3′ region 40′a that is hybridized to at least a portion of the second surface primer 41. In some embodiments, at least a portion of the 3′ region 40′a is from the adapter ligated to the first polynucleotide template 30a. For the example, the 3′ region 40′a may include a P5′ sequence that is configured to hybridize to the P5 sequence of the second surface primer 41. Unlike the single stranded surface sequencing methods of the present disclosure, the pre-sequencing complex 5 further includes a cleaved fourth polynucleotide template 30′b(c) having a free 5′ and a free 3′ end. The cleaved fourth polynucleotide 30′b(c) is hybridized to the first polynucleotide template 30a in a double stranded bridge structure.

In some embodiments, pre-sequencing complex 5 is provided as described in reference to FIG. 6 later herein.

In some embodiments the second surface primer 41 comprises at least a portion of a second surface oligonucleotide 40 (see FIG. 6) that was immobilized on the surface. As described elsewhere herein, the second surface oligonucleotide 40 may be cleaved to give the second surface primer 41.

In step M of FIG. 4, at least a portion of the first polynucleotide template 30a is sequenced as a first read region 31. Sequencing may include sequencing by synthesis where the second surface primer 41 is enzymatically extended in the 5′ to 3′ direction thereby creating a portion of a second polynucleotide template 30′a that is complementary to the first polynucleotide template 30a. The portion of the second polynucleotide template 30′a generated in the extension is the first read region 31. The enzymatic extension uses the first polynucleotide template 30a as the template and at least a portion of the second surface primer 41 as the sequencing primer. In contrast to the single strand surface sequencing methods of the present disclosure, the pre-sequencing complex 5, has a double stranded bridge structure. As the first read region is extended it displaces a 5′ portion of cleaved fourth polynucleotide 30′b(c). The displaced portion 30′b(cd) of the cleaved fourth polynucleotide may be referred to as a flap or an overhang.

In step N of FIG. 4, the second polynucleotide template 30′a is extended from the first read region 31 through the incorporation of nucleotides and the use of the first polynucleotide template 30a as the template. As the second polynucleotide 30′a grows, the cleaved fourth polynucleotide 30′b(c) is further displaced until eventual it is completely displaced from the first polynucleotide template to which it was previously hybridized. That is, the displaced portion 30′b(cd) grows in length until the displaced portion 30′b(cd) comprises the entire cleaved fourth polynucleotide 30′b(c). The nucleotides incorporated in step N may be blocked nucleotides, such as those used in SBS, or unblock nucleotides allowing for rapid chain extension. The second polynucleotide template 30′a is covalently bonded to the second surface primer 41 at its 5′ end and has a free 3′ end. The second polynucleotide template 30′a is complementary to the first polynucleotide template 30a and includes the first read region 31 proximate its 5′ end, and a 3′ end region 20′a that is complementary to at least a portion of the first surface oligonucleotide 20. The first polynucleotide template 30a and the second polynucleotide template 30′a are hybridized in a double stranded bridged structure.

Unlike the single strand sequencing methods of the present disclosure, the strand displacement sequencing methods only includes the first step of linearization; that is, the cleavage of one strand of a double stranded bridge structure. The cleaved strand is not removed prior to sequencing.

In step O of FIG. 4, the first surface oligonucleotide 20 is cleaved to produce a first surface primer 21 and a cleaved first polynucleotide template 30a(c). The free 3′ end of first surface primer 21 includes a terminal hydroxyl at the 3′ position on the deoxyribose. Various cleavage or cleavage and conversion methods may be used including, for example, abasic cleavage, chemical cleavage, cleavage of ribonucleotides, photochemical cleavage, hemimethylated DNA cleavage, nicking endonuclease cleavage, restriction enzyme cleavage as described elsewhere herein, or kinase or phosphatase conversion as described elsewhere herein.

In step P of FIG. 4, at least a portion of the second polynucleotide template 30′a is sequenced as a second read region 34. Sequencing includes sequencing by synthesis where the first surface primer 21 is enzymatically extended in the 5′ to 3′ direction thereby creating a portion of a third polynucleotide template 30c. The portion of the third polynucleotide template 30c generated in the extension is the second read region 34. The enzymatic extension uses the second polynucleotide template 30′a as the template and at least a portion of the first surface primer 21 as the sequencing primer. As the second read region 34 is extended it displaces a 5′ portion of the cleaved fourth polynucleotide 30a(c). The displaced portion 30a(cd) may be referred to as a flap or an overhang.

### Double Stranded Surface Sequencing Via Nick Translation (dsSurfSeqfnick Translation))

FIG. 5 is a schematic overview of a double stranded surface sequencing via nick translation method consistent with some embodiments of the present disclosure. Nick translation includes the removal of nucleotides from a 5′ end portion of a nicked strand of double stranded DNA and replacing the removed nucleotides with newly incorporated nucleotides on the 3′ end of a growing strand, which may be a primer. In nick translation the growing strand and the nicked strand form a double stranded structure with an unnicked strand. The growing strand and the nicked stranded are hybridized to the unnicked strand. The 3′ end of the growing strand and the 5′ end of the nicked strand are zero to fifty (e.g., zero to one, zero to two, zero to three, zero to four, zero to five, zero to ten, zero to fifteen, etc.) nucleotides apart. The 5′ end of the nicked strand and the 3′ end of the growing strand are considered to be zero nucleotides apart when only a nick, with no intervening gap or with no flap, in the strand separates 5′ end of the nicked strand and the 3′ end of the growing strand. A flap nuclease may remove the nucleotides from the 5′ end portion. As used herein, a “flap nuclease” is a nuclease that prevents formation of a flap or cleaves or removes at least a portion of a flap that is or would otherwise be formed due to the addition of nucleotides to the 3′ end of the growing strand. In some embodiments, the flap nuclease has 5′ to 3′ exonuclease activity. In some embodiments, the flap nuclease has endonuclease activity. In some embodiments, the flap nuclease having endonuclease or exonuclease activity recognizes a nick or break in a single strand of the double stranded DNA and introduces a nick in the nicked strand at a location 3′ of the recognized nick or break. A polymerase may be used to introduce the newly incorporated nucleotides using the 3′ end of a surface primer (e.g., 41) or growing strand (the 3′ hydroxyl at the location of the nick; e.g., 30′a) as a primer and using the unnicked strand as a template (e.g., 30a, 30a(c)). At least some of the newly incorporated nucleotides may be labeled or labeled and blocked so that the identity of the newly incorporated nucleotides may be determined through a sequencing process. This process (nicking and incorporation) may be continuous or may be repeated to create a strand that has a plurality of newly incorporated nucleotides.

Similar to the double stranded sequencing via displacement method of the present disclosure, double stranded surface sequencing via nick translation workflow includes providing a pre-sequencing complex 5. The pre-sequencing complex 5 includes a surface 15, a first surface oligonucleotide 20, a second surface primer 41, and a first polynucleotide template 30a. The first surface oligonucleotide 20 is bound to the surface 15 at its 5′ end. The second surface primer 41 is bound to the surface at its 5′ end and has a free 3′ end with a terminal hydroxyl at the 3′ position on the deoxyribose. The first polynucleotide template 30a is covalently bound to the first surface oligonucleotide 20 at its 5′ end. The first polynucleotide template includes a 3′ region 40′a that is hybridized to at least a portion of the second surface primer 41. In some embodiments, at least a portion of the 3′ region 40′a is from the adapter ligated to the first polynucleotide template 30a. For example, the 3′ region 40′a may include a P5′ sequence that is configured to hybridize to the P5 sequence of the second surface primer 41. Unlike the single stranded surface sequencing methods of the present disclosure, the pre-sequencing complex 5 further includes a cleaved fourth polynucleotide template 30′b(c) having a free 5′ and a free 3′ end. The cleaved fourth polynucleotide 30′b(c) is hybridized to the first polynucleotide template 30a in a double stranded bridge structure.

In some embodiments the second surface primer 41 comprises at least a portion of a second surface oligonucleotide 40 (see FIG. 6) that was immobilized on the surface. As described elsewhere herein, the second surface oligonucleotide 40 may be cleaved to give the second surface primer 41.

In some embodiments, the pre-sequencing complex 5 can be provided using pre-sequencing methods described later herein.

In step R of FIG. 5, at least a portion of the first polynucleotide template 30a is sequenced as the first read region 31. Sequencing may include sequencing by synthesis where the second surface primer 41 is enzymatically extended in the 5′ to 3′ direction thereby creating a portion of a second polynucleotide template 30′a that is complementary to the first polynucleotide template 30a. The portion of the second polynucleotide template 30′a generated in the extension is the first read region 31. The enzymatic extension uses the first polynucleotide template 30a as the template and at least a portion of the second surface primer 41 as the sequencing primer. Similar to the double stranded surface sequencing via displacement methods of the present disclosure, the pre-sequencing complex 5, has a double stranded bridge structure. In double stranded surface sequencing by nick translation, nucleotides on the strand ahead (cleaved fourth polynucleotide 30′b(c)) of the growing first read region 31 are removed via nick translation. Such nick translation may limit impedance of the growing the first read region 31.

To facilitate removal of the nucleotides on the impeding strand, a flap nuclease (i.e., a domain or protein having flap nuclease activity) may be added during certain steps of the method (e.g., during sequencing and/or chain extension that is independent from sequencing). As used herein, a “flap nuclease” is a protein, or domain thereof, that can introduce a break in, or remove nucleotides from, one strand of double-stranded DNA. A flap nuclease may be a flap nicking enzyme. In some embodiments, the flap nuclease comprises a domain of a protein than includes domains having other enzymatic activity.

The flap nuclease may have exonuclease or endonuclease activity. In some embodiments, the flap nuclease has exonuclease activity. In some embodiments, the flap nuclease has 5′ to 3′ exonuclease activity. The use of a flap nuclease having 5′ t o3′ exonuclease activity may allow for the sequential 5′ to 3′ removal of nucleotides on the impeding strand. In some embodiments, the flap nuclease has endonuclease activity. In some embodiments, the flap nuclease having endonuclease activity removes two or more nucleotides from the impeding strand simultaneously.

Any suitable flap nuclease may be used. A flap nuclease may be a flap nuclease that is found in nature or a synthetically evolved protein that is designed to have flap nuclease activity. Examples of naturally occurring flap nuclease includes full-length or small subunits from the PolA family of DNA polymerases such as Taq DNA polymerase (e.g., amino acids 1-305 or amino acids 1-292; Bst DNA polymerase (e.g., amino acids 1-304); Flap Endonuclease I (FEN1); GINS- associated nuclease (GAN); RecJ family of exonucleases; lambda exonuclease, and combinations thereof. Examples of evolved flap nucleases include, for example RecJF. Table 1 gives examples of flap nuclease. Although a specific organism is shown for some of the flap nuclease in Table 1, the same or similar flap nuclease may be isolated from a different organism. In some embodiments, the flap nuclease includes the Taq DNA polymerase or a portion thereof that has flap nuclease activity. In some embodiments, the flap nuclease includes the Bst DNA polymerase or a portion thereof that has flap nuclease activity. In some embodiments, the flap nuclease includes FEN1 or a portion thereof that has flap nuclease activity. In some embodiments, the flap nuclease includes GAN or a portion thereof that has flap nuclease activity.

TABLE 1

| | | | |
|---|---|---|---|
| Flap Nuclease | UniProt Number | Protein Data Bank Number or relevant reference | Available From |
| Taq DNA polymerase | P19821 | 1TAQ | New England Biolabs |
| Bst DNA polymerase | P52026 | 6MU4 | New England Biolabs |
| FEN1 | C5A639 (Thermococcus Gammatolerans) | | New England Biolabs |
| GAN | Q5JGL0 (Thermococcus Kodakarensis) | 5GHS | |
| GAN | NCBI number AHL22101 (Thermocuccus Nautili) | | |
| RecJ | Q5SJ47 ( Thermus Thermophilus) | 2ZXO | |
| Lambda exonuclease | P03697 | 4WUZ | New England Biolabs |
| RecJF | | Lovett, S.T., Kolodner, R.D. (1989). Proc. Natl. Acad. Sci. USA. 86, 2627-2631. | New England Biolabs |
| Bst 2.0 | | | New England Biolabs |

In embodiments of nick translation described herein, a flap nuclease is added at a step during each sequencing cycle. In embodiments, a flap nuclease is added after a number of sequencing cycles. In embodiments, a protein comprising polymerase activity for use in a sequencing cycle also comprises flap nuclease activity. In some embodiments, the protein comprising polymerase activity and a flap nuclease activity is a naturally occurring protein. In some embodiments, the protein comprising polymerase activity and a flap nuclease activity is a naturally occurring protein that has been modified to eliminate or reduce active domains that might otherwise interfere with a nick translation process described herein. In some embodiments, the protein comprising polymerase activity and a flap nuclease activity is a protein in which one or more domains comprising polymerase activity are coupled to one or more domains having flap nuclease activity. In some embodiments, the protein comprising polymerase activity and a flap nuclease activity is a fusion protein. In some embodiments, a flap nuclease may prevent the formation and/or remove an impeding strand flap (i.e., one or more nucleotides forming a single strand of DNA that has been displaced from the template strand). In some such embodiments, a flap nuclease is added during each SBS cycles. In some such embodiments for every SBS incorporation cycle where a nucleotide is added onto the growing read strand 31, a flap nuclease removes one or more nucleotide on the cleaved fourth polynucleotide 30′b(c). Thus, avoiding the formation of a displaced strand (i.e., a flap), which forms in the double stranded surface sequencing by displacement methods of the present disclosure.

In some embodiments, the flap nuclease is added after a number of SBS cycles. In such embodiments, a portion of the cleaved fourth polynucleotide 30′b(c) is displaced by the growing read strand 31 prior to cleavage and removal; that is, a small flap is allowed to form. For example, several cycles of SBS incorporation may be run via the double stranded sequencing via displacement methods of the present disclosure. After a predetermined number of SBS cycles where a flap has formed, a flap nuclease may be introduced to nick the cleaved fourth polynucleotide 30′b(c) such that the displaced portion of the cleaved fourth polynucleotide 30′b(c) is cleaved. A flap nuclease may be introduced at any suitable interval during the SBS process. For example, a flap nuclease may be introduced after or during every 2 SBS cycles, every 4 SBS cycles, every 6 SBS cycles, every 8 SBS cycles, every 10 SBS cycles, every 20 SBS cycles, every 30 SBS cycles, every 40 SBS cycles, every 50 SBS cycles, and so on.

In some embodiments of a nick translation method, a protein comprising DNA polymerase activity also includes flap nuclease activity. In some such embodiments, a flap nuclease is operably linked to a DNA polymerase forming a polymerase-flap nuclease construct. As used herein, the term “operably linked” refers to a direct or indirect covalent linking between the polymerase and the flap nuclease. Thus, a flap nuclease and a polymerase that are operably linked may be directly covalently coupled to one another. Conversely, a flap nuclease and a polymerase that are operably linked may be connected by mutual covalent linking to an intervening component (e.g., a flanking sequence or linker). Any suitable polymerase may be used in a polymerase-flap nuclease construct. Examples of suitable polymerase may be found in U.S. Pat. Application No. US16/703569 (US11001816B2), PCT Application Number PCT/US2013/03169 (WO2014142921A1), all of which is hereby incorporated by reference in its entirety. In some embodiments, the polymerase has strand displacing activity. In some embodiments, the polymerase does not have strand displacing activity.

The flap nuclease and the polymerase may be operably linked through one or more linkers. The term “linker” as used herein refers any bond, small molecule, peptide sequence, or other vehicle that covalently links flap nuclease and the polymerase. Linkers are classified based on the presence of one or more chemical motifs such as, for example, including a disulfide group, a hydrazine group or peptide (cleavable), or a thioester group (non-cleavable). Linkers also include charged linkers, and hydrophilic forms thereof as known in the art.

Suitable linkers for linking the flap nuclease and polymerase include a peptide linker such as a natural linker, an empirical linker, or a combination of natural and/or empirical linkers. Natural linkers are derived from the amino acid linking sequence of multi-domain proteins, which are naturally present between protein domains. Properties of natural linkers such as, for example, length, hydrophobicity, amino acid residues, and/or secondary structure can be exploited to confer desirable properties to a multi-domain compound that includes natural linkers connecting the flap nuclease and polymerase. In some embodiments, the linker is an empirical linker. In some embodiments, the empirical linkers comprises flexible linker, a rigid linker, or a cleavable linker. Flexible linkers can provide a certain degree of movement or interaction at the joined components. Flexible linkers typically include small, non-polar (e.g., Gly) or polar (e.g., Ser or Thr) amino acids, which provide flexibility, and allow for mobility of the connected components. Rigid linkers can successfully keep a fixed distance between the flap nuclease and the polymerase to maintain their independent functions, which can provide efficient separation of the flap nuclease and polymerase and/or sufficiently reduce interference between the flap nuclease and the polymerase. Examples of peptide linkers include GGGGSGGGGSGGGGS (SEQ ID NO. 5), AALGGAAAAAAS (SEQ ID NO. 6), and ALEEAPWPPPWGA (SEQ ID NO. 7).

In some embodiments, the natural linker or empirical linker is covalently attached to the polymerase, flap nuclease, or both, using bioconjugation chemistries. Bioconjugation chemistries are well known in the art and include but are not limited to, NHS-ester ligation, isocyanate ligation, isothiocyanate ligation, benzoyl fluoride ligation, maleimide conjugation, iodoacetamide conjugation, 2-thiopyridine disulfide exchange, 3-arylpropiolonitrile conjugation, diazonium salt conjugation, PTAD conjugation, and Mannich ligation.

In some embodiments, the natural linker or empirical linker, the flap nuclease, the polymerase, or any combinations thereof, may include one or more unnatural amino acids that allow for bioorthogonal conjugation reactions. As used herein, “bioorthogonal conjugation” refers to a conjugation reaction that uses one or more unnatural amino acids or modified amino acids as a starting reagent. Examples of bioorthogonal conjugation reactions include but are not limited to, Staudinger ligation, copper-catalyzed azide-alkyne cycloaddition, strain promoted [3+2] cycloadditions, tetrazine ligation, metal-catalyzed coupling reactions, or oxime-hydrazone ligations. Examples of non-natural amino acids include, but are not limited to, azidohomoalanine, 2 homopropargylglycine, 3 homoallylglycine, 4 p-acetyl-Phe, 5 p-azido-Phe, 3-(6-acetylnaphthalen-2-ylamino)-2-aminopropanoic acid, N^{ε}-(cyclooct-2-yn-1-yloxy)carbonyl)L-lysine, N^{ε}-2-azideoethyloxycarbonyl-L-lysine, Nε-p-azidobenzyloxycarbonyl lysine, Propargyl-L-lysine, or trans-cyclooct-2-ene lysine.

In some embodiments, the linker is derived from a small molecule, such as a polymer. Example polymer linkers include but are not limited to, poly-ethylene glycol, poly(N-isopropylacrylamide), and N,N′-dimethylacrylamide)-co-4-phenylazophenyl acrylate. The small molecule linkers generally include one or more reactive handles allowing conjugation to the polymerase, flap nuclease, or both. In some embodiments, the reactive handle allows for a bioconjugation or bioorthogonal conjugation. In some embodiments, the reactive handle allows for any organic reaction compatible with conjugating a linker to the polymerase, flap nuclease, or both.

The linker may be conjugated at any amino acid location of the polymerase, flap nuclease, or both. For example, the linker may be conjugated to the N-terminus, C-terminus, or any amino acid between of the flap nuclease, polymerase or both. In some embodiments, the linker is conjugated to the N terminus of the flap nuclease and the N terminus of the polymerase. In some embodiments, the linker is conjugated to the C terminus of the flap nuclease and the C terminus of the polymerase. In some embodiments, the linker is conjugated to the C terminus of the flap nuclease and the N terminus of the polymerase. In some embodiments, the linker is conjugated to the N terminus of the flap nuclease and the C terminus of the polymerase.

In embodiments, where the flap nuclease and polymerase are operably coupled by a peptide linker, the flap nuclease-polymerase construct may be referred to as a fusion protein or a flap nuclease-polymerase (or polymerase-flap nuclease) fusion. Fusion proteins such as a flap nuclease-polymerase fusion can be produced by expression in a host cell (e.g., recombinant expression).

In some embodiments, the flap nuclease-polymerase construct includes the Taq DNA polymerase or a portion thereof that has flap nuclease activity. In some embodiments, the flap nuclease-polymerase construct includes the Bst DNA polymerase or a portion thereof that has flap nuclease activity. In some embodiments, the flap nuclease-polymerase construct includes FEN1 or a portion thereof that has flap nuclease activity. In some embodiments, the flap nuclease-polymerase construct includes GAN or a portion thereof that has flap nuclease activity. In some embodiments, the flap nuclease-polymerase includes the Bst DNA polymerase or a portion thereof that has flap nuclease activity and the linker includes the sequence of SEQ ID NO. 5, SEQ ID NO. 6, or SEQ ID NO. 7. In some embodiments, the flap nuclease-polymerase includes the Taq DNA polymerase or a portion thereof that has flap nuclease activity and the linker includes the sequence of SEQ ID NO. 5, SEQ ID NO. 6, or SEQ ID NO. 7. In some embodiments, the flap nuclease-polymerase includes FEN1 or a portion thereof that has flap nuclease activity and the linker includes the sequence of SEQ ID NO. 5, SEQ ID NO. 6, or SEQ ID NO. 7. In some embodiments, the flap nuclease-polymerase includes GAN or a portion thereof that has flap nuclease activity and the linker includes the sequence of SEQ ID NO. 5, SEQ ID NO. 6, or SEQ ID NO. 7.

In step S of FIG. 5, the second polynucleotide template 30′a is extended from the first read region 31 through the incorporation of nucleotides and the use of the first polynucleotide template 30a as the template. During this step, nick translation enzymes may be present throughout, may be periodically introduced, or may be omitted. If nick translation enzymes are omitted, the remaining portion of the cleaved fourth polynucleotide 30′b(c) can be completely displaced from the first polynucleotide template 30a to which it was previously hybridized. The nucleotides incorporated may be blocked nucleotides, such as those used in SBS, or unblock nucleotides allowing for rapid chain extension. The second polynucleotide template 30′a is covalently bonded to the second surface primer at its 5′ end and has a free 3′ end. The second polynucleotide template 30′a is complementary to the first polynucleotide template 30a and includes the first read region 31 proximate the 5′ end, and a 3′ end region 20′a that is complementary to at least a portion of the first surface oligonucleotide 20. The first polynucleotide template 30a and the second polynucleotide template 30′a are hybridized in a double stranded bridged structure.

Unlike the single strand sequencing methods of the present disclosure, the double stranded surface sequencing method via nick translation only requires the first step of linearization, that is, the cleavage of one strand of a double stranded bridge structure. The cleaved strand is not removed prior to sequencing.

In step T of FIG. 5, the first surface oligonucleotide 20 is cleaved to produce a first surface primer 21 and a cleaved first polynucleotide template 30a(c). The free 3′ end of the first surface primer 21 includes a terminal hydroxyl at the 3′ position on the deoxyribose. Various cleavage or cleavage and conversion methods may be used including, for example, abasic cleavage, chemical cleavage, cleavage of ribonucleotides, photochemical cleavage, hemimethylated DNA cleavage, nicking endonuclease cleavage, and restriction enzyme cleavage as described elsewhere herein, and kinase or phosphatase conversion as described elsewhere herein.

In step U of FIG. 5, at least a portion of the second polynucleotide template 30′a is sequenced as the second read region 34. Sequencing may include sequencing by synthesis where the first surface primer 21 is enzymatically extended in the 5′ to 3′ direction thereby creating a portion of a third polynucleotide template 30c that is complementary to the second polynucleotide template 30′a. The portion of the third polynucleotide template 30c generated in the sequencing extension is the second read region 34. The enzymatic extension uses the second polynucleotide template 30′a as the template and at least a portion of the first surface primer 21 as the sequencing primer. In double stranded sequencing by nick translation, the nucleotides on the strand ahead of the growing first read region (cleaved first polynucleotide template 30′a(c)) are removed via nick translation. Suitable accessory nick translation enzymes and procedures are described elsewhere herein.

FIG. 2 is a flow chart illustrating an overview of an embodiment of a pre-sequencing workflow method consistent with the present disclosure. The method includes providing a surface, a first surface oligonucleotide bound to the surface at its 5′ end, a second surface oligonucleotide bound to the surface at its 5′ end and having a free 3′ end, a first polynucleotide template bound to the 3′ end of the first surface oligonucleotide and having a free 3′ end, and a fourth polynucleotide template that is complementary to the first polynucleotide template that has a free 3′ end and is bound to the 3′ end of the second surface polynucleotide (700). At least a portion of the first polynucleotide template, in proximity to its free 3′ end, is hybridized to at least a portion of the second surface oligonucleotide. At least a portion of the fourth polynucleotide template in proximity to its free 3′ end is hybridized to at least a portion of the first surface oligonucleotide. The method further includes cleaving the second surface oligonucleotide or a portion of the fourth polynucleotide, such as a portion of an adapter region, to produce the second surface primer bound to the surface at the 5′ end and having a free 3′ end and to produce a cleaved fourth polynucleotide template having a free 5′ end and a free 3′ end (800). The method may optionally include hybridizing at least a portion, in proximity to the free 3′ end of the first polynucleotide template, to at least a portion of the second surface primer (900), if the portion, in proximity to the free 3′ end of the first polynucleotide template, is not already hybridized to at least a portion of the second surface primer.

FIG. 6 is a schematic overview of a pre-sequencing method consistent with the embodiments of the present disclosure. FIG. 6 depicts the workflow for generation of a pre-sequencing complex 10 that may be used in the single strand surface sequencing methods of the present disclosure. Pre-sequencing complex 5 is an intermediate in the workflow to create pre-sequencing complex 10. Pre-sequencing complex 5 may be used in the double stranded surface sequencing via displacement (FIG. 4) and double stranded surface sequencing via nick translation (FIG. 5) methods of the present disclosure.

In the depicted workflow, complex 1 is provided. Complex 1 includes the surface 15, the first surface oligonucleotide 20, a second surface oligonucleotide 40, the first polynucleotide template 30a, and a fourth polynucleotide template 30′b. The first surface oligonucleotide 20 and the second surface oligonucleotide 40 are attached to a surface 15 at their respective 5′ ends. The 5′ end of the first polynucleotide template 30a is covalently bound to the 3′ end of the first surface oligonucleotide 20. The first polynucleotide template includes a 3′ region 40′a that is annealed to at least a portion of the second surface oligonucleotide 40. The 5′ end of a fourth polynucleotide template 30′b is covalently bound to the 3′ end of the second surface oligonucleotide 40. The second polynucleotide template includes a 3′ region 20′b that is annealed to at least a portion of the first surface oligonucleotide 20. The first polynucleotide template 30a is hybridized to the fourth polynucleotide template 30′b in a double stranded bridged structure.

Steps X and Y of FIG. 6 illustrate the process of linearization. Linearization includes the cleavage of one strand of a double stranded bridged structure (Step X). Linearization also includes removing the polynucleotide strand that is no longer covalently attached to the surface (Step Y). Methods and reagents for accomplishing linearization are described elsewhere herein.

Briefly, in step X of FIG. 6, the second surface oligonucleotide 40 (or a portion of the fourth polynucleotide template 30′b, such as an adapter portion) is cleaved to give a second surface primer 41 and a cleaved fourth polynucleotide template 30′b(c). The resulting complex is pre-sequencing complex 5 that can be used for the double stranded surface sequencing via displacement and the double stranded surface sequencing via nick translation methods of the present disclosure. The free 3′ end of the second surface primer 41 has a terminal hydroxyl at the 3′ position on the deoxyribose. Various cleavage methods may be used including, for example, abasic cleavage, chemical cleavage, cleavage of ribonucleotides, photochemical cleavage, hemimethylated DNA cleavage, nicking endonuclease cleavage, and restriction enzyme cleavage as described elsewhere herein.

In some embodiments, abasic cleavage is used. In such embodiments, the second surface oligonucleotide 40 (or a portion of the fourth polynucleotide template 30′b, such as an adapter portion) has a second excisable base 42 (the first excisable base 22 being a part of the first polynucleotide template 30a or the first surface oligonucleotide 20). In embodiments where the first polynucleotide template 30a or the first surface oligonucleotide 20 do not include an excisable base, the excisable base of the second surface oligonucleotide 40 or fourth polynucleotide template 30′b may be the first excisable base. Stated differently, “first” and “second” are used for clarity and are not meant to imply more than one excisable base. In some embodiments, cleavage is accomplished by removing the second excisable base 42 creating an abasic site and subsequent cleavage of the abasic site to give cleaved the fourth polynucleotide template 30′b(c) and a cleaved second surface oligonucleotide 40c that has a terminal phosphate group (step X(1)). In some embodiments, the cleaved second surface oligonucleotide 40c is treated to convert the terminal phosphate group to a terminal 3′ hydroxyl group (step X(2)). Reagents and procedures for abasic cleavage and conversion of the terminal phosphate group to a 3′ hydroxyl group are described elsewhere herein.

In step Y of FIG. 6, the cleaved fourth polynucleotide template 30′a(c) is removed through denaturation methods and optional washing described elsewhere herein resulting in pre-sequencing complex 10.

In some embodiments, the surface including a plurality of pre-sequencing complex 10 (or complex 5 if double stranded sequencing is performed) is treated with an exonuclease. The exonuclease will remove at least a portion of surface oligonucleotides that are not participating in a double stranded bridged structure of pre-sequencing complex 10. The exonuclease may completely remove individual surface oligonucleotides or remove portions of individual surface oligonucleotides. Treating the surface with an exonuclease prior to applying the sequencing methods of the present disclosure may result in a lower background signal.

The methods described herein allow for sequencing of template polynucleotides using surface primers. Accordingly, separate sequencing primers may not be needed as reagents for sequencing.

In some embodiments, a kit comprises all reagents needed for sequencing polynucleotides according to the methods described herein. The kit may be free of sequencing primers. Any of the reagents disclosed herein may be included in the kit. For example, the kit may include a polymerase and labeled, blocked nucleotides. The kit may include unblocked nucleotides for extension, for example, after the first sequencing read. The kit may include a cleavage reagent and, if needed, a conversion reagent as described herein. The kit may include any or all reagents needed to accomplish chemical cleavage such as for example, OsO₄ or precursor compounds used to generate OsO₄ in situ, K₃[Fe(CN)₆], peroxide, N-methylmorpholine N-oxide (NMO), I₂, silver salts, or any combination thereof. The kit may include reagents to carry out the pre-sequencing methods described herein. For example, the kit may comprise enzymes and nucleotides for amplification and cluster formation. The kit may comprise an exonuclease to remove surface oligonucleotides on which clusters were not formed. The kit may comprise a flap nuclease or a polymerase-flap nuclease construct for use in embodiments of double stranded surface sequencing methods.

Throughout this application, various publications are referenced. The disclosures of these publications in their entireties are hereby incorporated by reference into this application.

### EXAMPLES OF EMBODIMENTS

The invention is defined in the claims. However, below there is provided a non-exhaustive listing of non-limiting examples of embodiments. Any one or more of the features of these aspects may be combined with any one or more features of another example, embodiment, or aspect described herein.

Embodiment 1. Embodiment 1 is A sequencing method comprising:
(a) providing a surface, a first surface oligonucleotide bound to the surface at a 5′ end, a second surface primer bound to the surface at a 5′ end and having a free 3′ end, a first polynucleotide template covalently bound to the 3′ end of the first surface oligonucleotide, the first polynucleotide template comprising a free 3′ end, wherein at least a portion of the first polynucleotide template in proximity to the free 3′ end is hybridized to at least a portion of a second surface primer;
(b) sequencing at least a portion of the first polynucleotide template by extending the second surface primer from the free 3′ end using the first polynucleotide template as a template and at least a portion of the second surface primer as a primer, thereby generating a second polynucleotide template covalently bound to the second surface primer and having a free 3′ end, the second polynucleotide template complementary to the first polynucleotide template and complementary to at least a portion of the first surface oligonucleotide in proximity to the free 3′ end, the second polynucleotide template comprising a first read region;
(c) cleaving the first surface oligonucleotide or a 5′ portion of the first polynucleotide template to produce a first surface primer bound to the surface at the 5′ end and having a free 3′ end and to produce a cleaved first polynucleotide template having a free 5′ end and a free 3′ end; and
(d) sequencing at least a portion of the second polynucleotide template by extending the first surface primer from the free 3′ end using the second polynucleotide template as a template and at least a portion of the first surface primer as a primer, thereby generating a third polynucleotide template that is complementary to the first polynucleotide template, the third polynucleotide template comprising a second read region.

Embodiment 2. Embodiment 2 is the method of embodiment 1, wherein step (a) further comprises:
providing a fourth polynucleotide template complementary to the first polynucleotide template and covalently bound to the 3′ end of the second surface oligonucleotide, the fourth polynucleotide template comprising a free 3′ end, wherein at least a portion of the fourth polynucleotide template in proximity to the free 3′ end is hybridized to at least a portion of the first surface oligonucleotide; and
cleaving the second surface oligonucleotide or a 5′ portion of the fourth polynucleotide template to produce the second surface primer and a cleaved fourth polynucleotide template having a free 5′ end and a free 3′ end.

Embodiment 3. Embodiment 3 is the method of embodiment 1, wherein cleaving the second surface oligonucleotide or a 5′ portion of the fourth polynucleotide template further comprises:
removing a first excisable base generating a cleaved second surface oligonucleotide; and
generating a hydroxyl at the free 3′ end of the cleaved second surface oligonucleotide to give the second surface primer; or
treating the surface with one or more dihydroxylation reagents to produce the second surface primer.

Embodiment 4. Embodiment 4 is the method of embodiment 2 or 3, wherein the second surface oligonucleotide or the 5′ portion of the fourth polynucleotide template comprises and allyl-dNTP and the method comprises treating the surface with one or more dihydroxylation reagents to produce the second surface primer.

Embodiment 5. Embodiment 5 is the method of embodiment 3 or 4, wherein the one or more dihydroxylation reagents comprises a single reagent comprising OsO4.

Embodiment 6. Embodiment 6 is the method of any one of embodiments 1 through 5, further comprising providing a cleaved fourth polynucleotide template have a free 5′ end and a free 3′ end, wherein the cleaved fourth polynucleotide template is hybridized to at least a portion of the first polynucleotide template.

Embodiment 7. Embodiment 7 is the method of any one of embodiments 1 through 6, wherein extension of the second surface primer from the free 3′ during sequencing of at least the portion of the first polynucleotide template results in displacement of at least a 5′ portion of the cleaved fourth polynucleotide template from the first polynucleotide template.

Embodiment 8. Embodiment 8 is the method of any one of embodiments 1 through 7, wherein sequencing at least a portion of the first polynucleotide template further comprises: removing nucleotides and/or polynucleotides from the cleaved fourth polynucleotide template thereby shortening the cleaved fourth polynucleotide template.

Embodiment 9. Embodiment 9 is the method of embodiment 8, wherein the nucleotides and/or polynucleotides are removed by a flap nuclease.

Embodiment 10. Embodiment 10 is the method of any one of embodiments 1 through 9, wherein a polymerase is used for the sequencing step (d) and wherein the polymerase is operably linked to the flap nuclease in a polymerase-flap nuclease construct.

Embodiment 11. Embodiment 11 is the method of embodiment 9 or 10, wherein the polymerase-flap nuclease construct comprises Taq DNA polymerase, Bst DNA polymerase GAN, FEN1, or a portion thereof that has flap nuclease activity.

Embodiment 12. Embodiment 12 is the method of any one of embodiments 1 through 11, further comprising denaturing the cleaved fourth polynucleotide template from the first polynucleotide template and washing the surface to remove the cleaved fourth polynucleotide template prior to sequencing at least the portion of the first polynucleotide template.

Embodiment 13. Embodiment 13 is the method of any one of embodiments 1 through 12, wherein cleaving the first surface oligonucleotide or a 5′ portion of the first polynucleotide template further comprises:
removing a second excisable base generating a cleaved first surface oligonucleotide; and
generating a hydroxyl at the free 3′ end of the cleaved first surface oligonucleotide to give the second surface primer.

Embodiment 14. Embodiment 14 is the method of any one of embodiments 1 through 13, further comprising denaturing the cleaved first polynucleotide template from the third polynucleotide template and washing the surface to remove the cleaved first polynucleotide template prior to sequencing at least the portion of the second polynucleotide template.

Embodiment 15. Embodiment 15 is the method of any one of embodiments 1 through 14, wherein step (a) further comprising treating the surface with an exonuclease.

Embodiment 16. Embodiment 16 is a kit comprising one or more of the reagents needed for sequencing at least the portion the first polynucleotide template and at least the portion of the second polynucleotide template according to the method of any one of embodiments 1 through 15, wherein the kit is free of sequencing primers. In some embodiments, the kit comprises all the reagents needed for sequencing at least the portion of the first polynucleotide template and at least a portion of the second polynucleotide template according to the methods of any one of embodiments 1 through 15.

Embodiment 17. Embodiment 17 is the kit of embodiment 16, wherein the reagents include a polymerase and labeled, blocked nucleotides.

Embodiment 18. Embodiment 18 is the kit of embodiment 16 or embodiment 17, wherein the reagents comprise a cleavage reagent.

Embodiment 19. Embodiment 19 is the kit of any one of embodiments 16 through 18, further comprising one or more reagents for amplifying template polynucleotides on a surface.

Embodiment 20. Embodiment 20 is the kit of anyone of embodiments 16 through 19, wherein the reagents comprise a flap nuclease.

### EXAMPLES

The polymerases used in the examples can be found in U.S. Provisional Pat. Application Number 63/412,241 (Pol(A)); U.S. Pat. Application No. US16/703569 (US11001816B2) (Pol(X)), and PCT Application Number PCT/US2013/03169 (WO2014142921A1) (Pol(z)).

### EXAMPLE 1: Single Stranded Surface Sequencing (ssSurfSeq)

### Materials and Methods

### General Protocol for ssSurfSeq

A standard MiniSeq reagent cartridge and flowcell were used for modified MiniSeq sequencing runs (Illumina, San Diego, CA). The library was loaded onto the sequencer using standard library denaturation and dilution conditions. Next, standard random flowcell bridge amplification was used to make clusters. After cluster formation, an exonuclease was used to remove excess surface primers. Next, the BLM1 reagent containing USER (New England Biolabs Inc, Ipswich, MA) was used to linearise the P5 surface primers. The standard sequencing by synthesis reagents were primed and the USER cleaved sites were deprotected using the standard deprotection method. In a standard run, the deprotection step is usually the first part of the paired end (PE) turn, but in ssSurfSeq the deprotection step is being used to turn the 3′ phosphate on the surface P5 primers to 3′ OH. The standard steps for the 1st base and SBS cycles of read 1 are done at 60° C. After read 1, the modified sequencing run calls for a custom PE turn. The first step in the custom PE turn follows the standard 12 cycles of PE turn resynthesis. However, in this case, the BMS reagent (polymerase and dNTPs) pumped at each cycle of the PE turn resynthesis extends the first read strand from final fully functional nucleotide of the read 1 to fill in the rest of the read 1 strand with dNTPs. An exonuclease treatment is then followed by R2 linearization using BLM2. BLM2 contains FpG and cleaves at the 8-oxo-G site within the P7 surface primers. A deprotection step is done which converts the 3′ phosphate left by the FpG enzyme to 3′ OH. After deprotection the standard steps are used for 1st and SBS cycles of read 2 at 60° C.

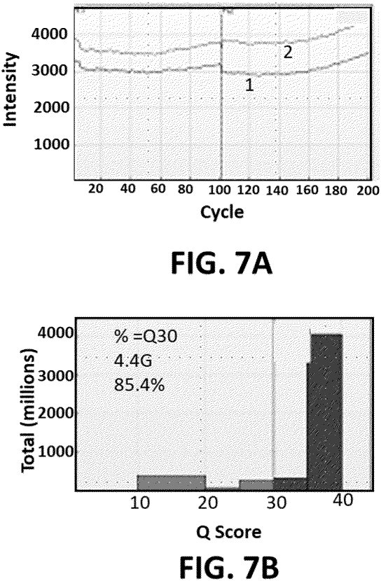

### ssSurfSeq Protocol Used to Generate the Data in FIGS. 7A-7B

The general ssSurfSeq protocol was GINS- associated nuclease used with the following changes. The standard PhiX control library (Illumina, Ca) was used at a final concentration of 1.8 pM. Prior to sequencing the first read, 48 dark cycles of synthesis by sequence incorporation followed by cleavage without imaging were done to skip reading the indexes and spacer sequences. Prior to sequencing the second read, 45 dark cycles of SBS incorporation followed by cleavage without imaging were done to skip reading the indexes and spacer sequences.

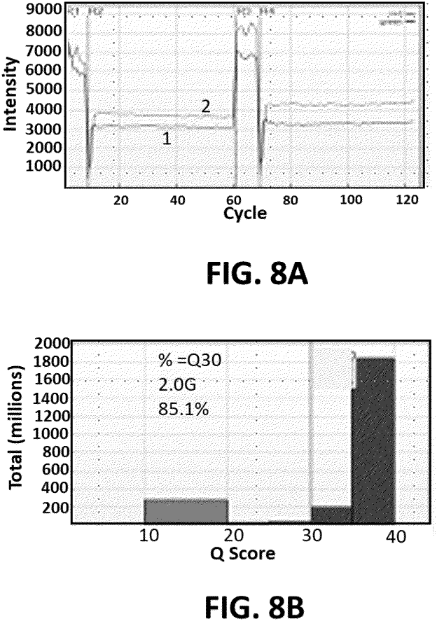

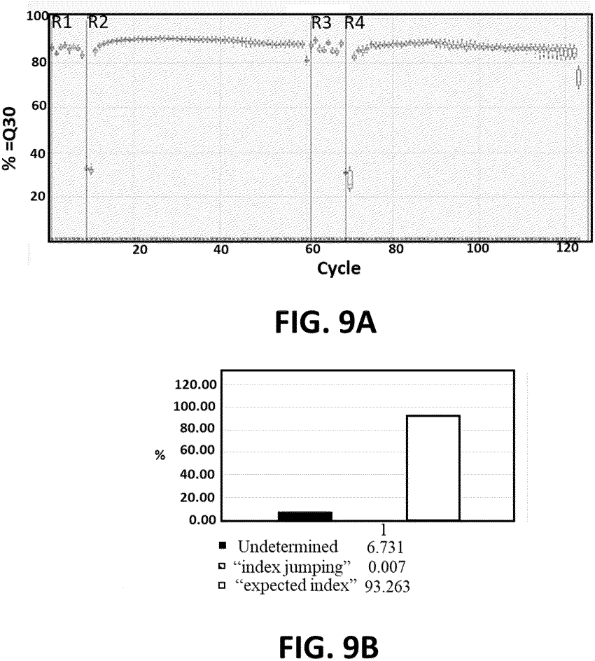

### ssSurfSeq Protocol Used to Generate the Data in FIGS. 8A-8B and 9A-9B

The general ssSurfSeq protocol was used with the following changes. A multiplex pool of TruSeq Nano PhiX libraries (Illumina, CA) was used at 1.8 pM final concentration. The multiplex pool contains 8 PhiX libraries with 8 unique dual indexes. Prior to sequencing the first read, a first indexing read is accomplished via SBS at 45° C. instead of the usual 60° C. Following the first indexing read, 33 dark cycles of synthesis by sequence incorporation followed by cleavage without imaging are done to skip reading spacer sequences prior to sequencing the first read. After the custom PE turn described in the general ssSurfSeq protocol, the second indexing read is accomplished via SBS at 45° C. Following the second indexing read, 34 dark cycles of synthesis by sequence incorporation followed by cleavage without imaging are done to skip spacer sequences prior to sequencing the second read.

### Results and Discussion:

FIGS. 7A-B, 8A-B, and 9A-B illustrate the quality of the sequencing data obtained using the ssSurfSeq. The charts in FIGS. 7A-7B show that ssSurfSeq was successfully used to sequence target nucleic acids by using dark cycling to skip spacer sequences and index sequences. The charts in FIGS. 8A-B and 9A-B show that ssSurfSeq was successfully used to sequence target nucleic acids and indexes.

FIGS. 7A and 8A are data by cycle plots showing the intensity per cycle during sequencing for a first and second reads. Additionally, FIGS. 7B and 8B are Q scores distribution plots that indicate the accuracy of the sequencing. Specifically, Q scores are used to estimate the accuracy of base calling during sequencing. Q is defined as -10×log10(e) where e is the estimated probability of the base call being wrong. Higher Q scores indicate a small probability of error. Generally, Q scores above 30 indicate high accuracy.

FIG. 7B, FIG. 8B, and FIG. 9A show that the ssSurfSeq method results in high Q scores when the index sequences are skipped via dark cycling (FIG. 7B) and when the index sequences are sequenced (FIGS. 8B, 9A). The percentage of base calls with a Q score greater than 30 is comparable when the index sequences are skipped via dark cycling (FIG. 7B, 85.4%) and when the index sequences are sequenced (FIG. 8B, 85.1%).

FIG. 7A and FIG. 8A show that ssSurfSeq method results in high signal intensity during sequencing both when the index sequences are skipped via dark cycling (FIG. 7A) and when the index sequences are sequenced (FIGS. 8A, 9A). The indexing reads (cycles ~1-10 and ~60-70) resulted in higher signal intensity than the nucleic acid target sequence reads (FIG. 8A). The target nucleic acid read intensities are comparable in both FIG. 7A and FIG. 8A.

FIG. 9B shows that the indexing reads were successful to give good demultiplexing results.

### EXAMPLE 2: Double Stranded Surface Sequencing via Displacement (dsSurfSeq(Displacement)) and Double stranded Surface Sequencing via Nick Translation (dsSurfSeq(Nick Translation))

### Materials and Methods

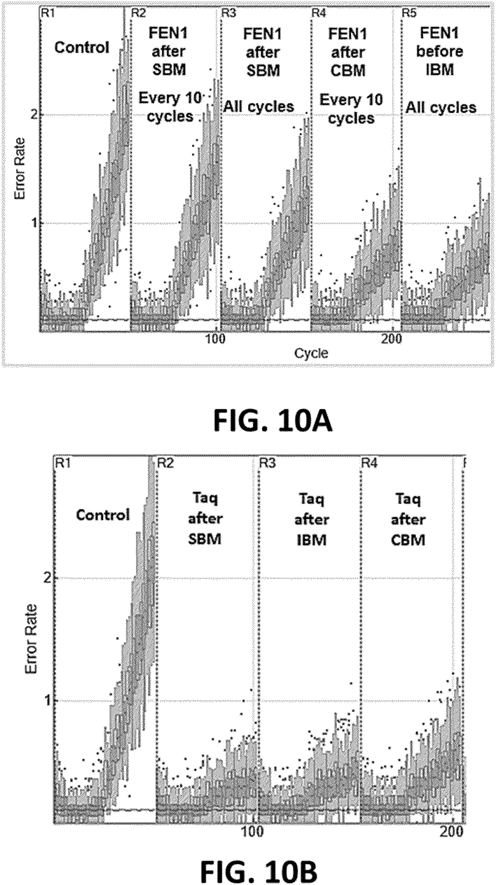

### Protocol Used to Generate Data in FIGS. 10A-B and 11.

Adapter sequences containing P5-BssSI-BspQI and P7 were ligated to fragmented human DNA mixed with 1% PhiX DNA to prepare the library. BssSI indicates the cleavage sequence for Nb.BssSI nickase (nicking endonuclease) and BspQI indicates the cleavage sequence for Nt.BspQI (nicking endonuclease) (both from New England Biolabs, MA).

Library molecules were clustered on a MiniSeq instrument using standard workflow. After clustering, the surface was treated with Exonuclease I and ends were repaired. All free 3′ ends were further blocked by addition of a blocking mix containing ddNTPs and a mixture of DNA polymerases. For nicking, Nt.BspQI was added to the surface to generate a free 3′OH after the adapter region (no dark cycles are required for this library as the cleavage occurs right after the adapter region). The read one (R1) SBS was performed from this free 3′ end for 51 cycles in double-stranded format. For all consecutive reads (R2-R5), the cluster was first cleaved with Nb.BssSI to allow the removal of SBS strand and generate a new priming site on the surface primer. The 3 cycles of amplification were performed using MiniSeq standard workflow. This resets the cluster to the initial condition allowing for comparison of different treatments on the same flow cell. It is worth mentioning that the process of “cluster generation” is only used for comparison of various double stranded sequencing methods and is not a necessary part of double stranded sequencing workflow. Although, it is possible to use this capability for certain applications if resequencing of the same cluster may be required.

The text within each read section indicates the modification to the SBS cycle chemistry. Control indicates no change to the SBS cycle chemistry resulting in double stranded SBS via strand displacement. All other conditions in FIGS. 10A and 10B show various degrees of nick translation that improves the accuracy of SBS compared to the control (dsSBS (displacement)). For instance, “FEN1 after SBM Every 10 cycles” indicates that a push of FEN1 reaction mix was added to the flow cell after the addition of the SBM (scan mix, from standard Illumina, Inc. cartridge) to the flow cell once every 10 cycles. Or Taq after CBM (cleavage mix, from standard Illumina, Inc. cartridge), means Taq DNA polymerase reaction mix was added to the flow cell after the CBM step at every cycle of the SBS.

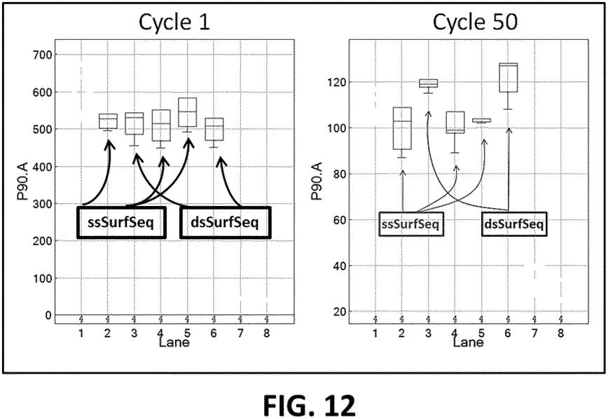

### Protocol Used to Generate Data in FIG. 12

For the dsSurfSeq(nick translation) a similar protocol to the previously described protocol was used with the following changes. A HighSeqX platform was used. The library was from fragmented human genomic DNA that was ligated directly to P5/P7 adapter sequences (the adapter sequences do not include SBS primer sites). After cluster formation, nicking was accomplished with USER (New England Biolabs, MA). The 3′ OH groups were deblocked using T4 PNK, and 7 dark cycles were run prior to sequencing the first read. To induce nick translation, Taq DNA polymerase was added in the Scan Mix to the flow cell to induce nick translation every cycle.

### Results and Discussion

FIGS. 10A and 10B are plots illustrating the error rate of dsSurfSeq(displacement) control, dsSurfSeq(nick translation) using FEN1 as a flap nuclease after every 10 cycles, and dsSurfSeq(nick translation) using Taq DNA polymerase after every cycle.

FIG. 10A shows that dsSurfSeq(nick translation) with FEN1 reduces the sequencing error rate compared to the dsSurfSeq(displacement) control. Additionally, FIG. 10B shows that dsSurfSeq(nick translation) with Taq polymerase significantly reduces the sequencing error rate compared to the dsSurfSeq(displacement) control.

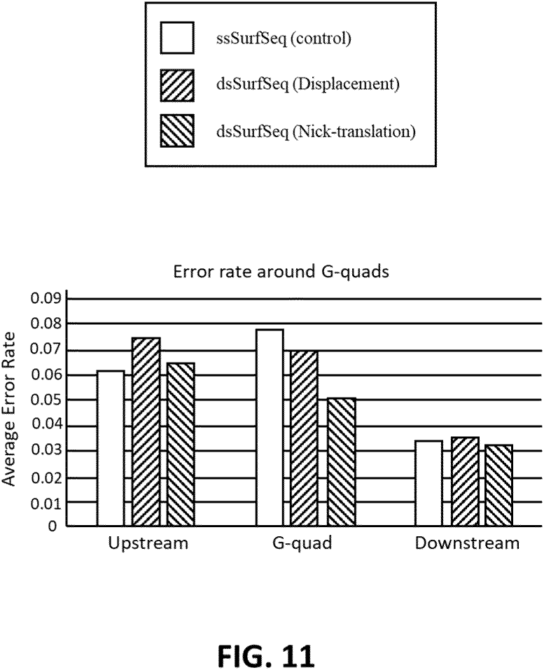

FIG. 11 shows that both dsSurfSeq(displacement) and dsSurfSeq(nick translation) reduce the error rate over G-quad sequences compared to the ssSurfSeq control. The error rate appears to be relatively high upstream to G-quad sequences. This value appears to be similar between ssSurfSeq and dsSurfSeq(nick translation), but higher in dsSurfSeq(displacement). Downstream to G-quad sequences the error rates are similar across sequencing conditions.

FIG. 12 shows data for the signal intensity at cycle 1 and after 50 cycles of sequencing using dsSurfSeq and ssSurfSeq on the same flow cell. The dsSurfSeq shows a higher signal intensity compared with ssSurfSeq controls. Thus, the data indicates that a lower signal decay may be achieved using dsSurfSeq(nick translation) in comparison to ssSurfSeq.

### EXAMPLE 3: Development of a Polymerase-flap Nuclease Fusion Protein for Use with dsSurfSeq(Nick Translation)

Various polymerase-flap nuclease fusion proteins were designed, synthesized, and tested for their ability to perform dsSurfSeq(nick translation). The flap nuclease-linker constructs are shown in Table 2. See Table 1 for the UniProt reference number, NCBI reference number, and/or PDB reference number that may be used to find the sequence information of the flap nuclease domain. Each construct was fused to polymerase X (Pol(X)).

TABLE 2

Polymerase-flap nuclease constructs

| | | | |
|---|---|---|---|
| Flap nuclease Domain | Host of nuclease | Linker Sequence | ID |
| GAN | T. kodakarensis | GGGGSGGGGSGGGGS | GAK GS |
| GAN | T. kodakarensis | AALGGAAAAAAS | GAK Helx |
| GAN | T. kodakarensis | ALEEAPWPPPWGA | GAK TaqL |
| GAN | T. nautili | GGGGSGGGGSGGGGS | GAN GS |
| GAN | T. nautili | AALGGAAAAAAS | GAN Helix |
| GAN | T. nautili | ALEEAPWPPPWGA | GAN TaqL |
| FEN1 | T. nautili | GGGGSGGGGSGGGGS | FEN GS |
| FEN1 | T. nautili | AALGGAAAAAAS | FEN Helix |
| FEN1 | T. nautili | ALEEAPWPPPWGA | FEN TaqL |
| Taq DNA Pol (1-305) | T. aquaticus | - | |
| Taq DNA Pol (1-305) | T. aquaticus | GGGGSGGGGSGGGGS | TaqFL-GS |
| Taq DNA Pol (1-305) | T. aquaticus | AALGGAAAAAAS | TaqFL-Helix |
| Taq DNA Pol (1-292) | T. aquaticus | GGGGSGGGGSGGGGS | TaqTR GS |
| Taq DNA Pol (1-292) | T. aquaticus | AALGGAAAAAAS | TaqTR Helix |
| Bst DNA Pol (1-301) | B. stearothermophilus | - | BstFL |
| Bst DNA Pol (1-301) | B. stearothermophilus | GGGGSGGGGSGGGGS | BstFL GS |
| Bst DNA Pol (1-301) | B. stearothermophilus | AALGGAAAAAAS | BstFL Helix |
| Bst DNA Pol (1-294) | B. stearothermophilus | GGGGSGGGGSGGGGS | B stTR-GS |
| Bst DNA Pol (1-294) | B. stearothermophilus | AALGGAAAAAAS | BstTR Helix |

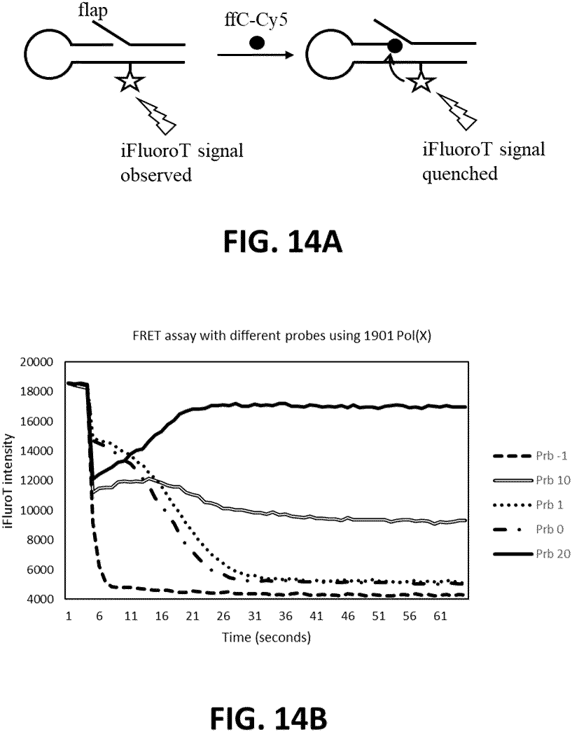

Various probes were designed to block the incorporation site on the hairpin to varying degrees allowing for assessment of incorporation kinetics of SBS in various stages of double stranded sequencing. A fluorescence resonance energy transfer (FRET) kinetic assay depicted in FIG. 14A, was used to evaluate the ability of polymerase X (Pol(X)) to incorporate a nucleotide into various double stranded hairpin probes (Prb) each having a different gap or nucleotide (nt) flap length (Prb0 = nick, no flap; Prb1 = 1 nt gap, no flap; Prb1 = 1 nt flap; Prb10 = 10 nt flap; Prb20 = 20 nt flap). The probes included an iFluroT nucleotide located on the opposite strand of the nick, gap, or flap. Upon incorporation of a ffC-Cy5 nucleotide, the iFluoroT emission is transferred to the Cy5 dye resulting in the quenching of the iFluoroT signal and the Cy5 FRET signal. FIG. 14B shows the results. Incorporation of the ffC-Cy5 gets progressively slower as the flap length increases.

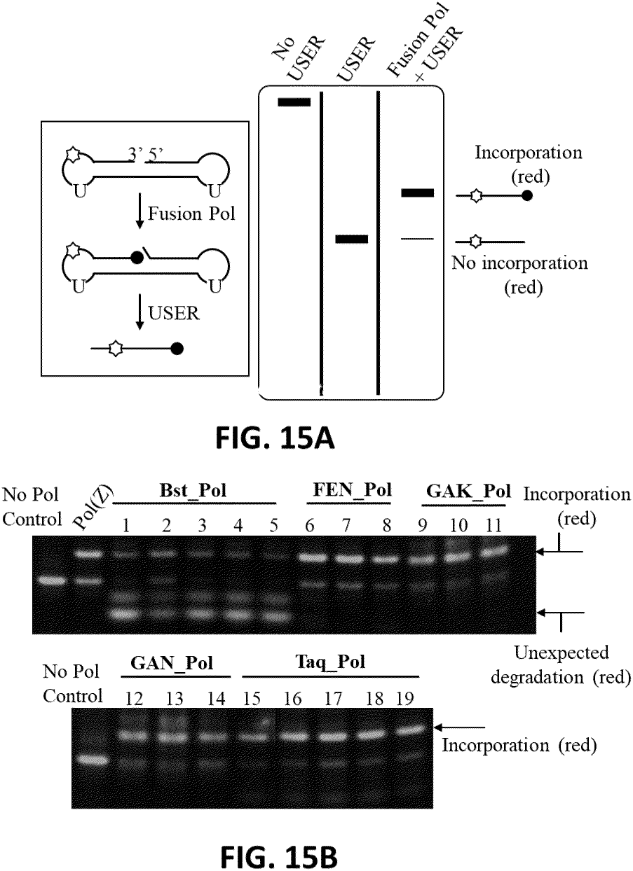

The ability of the flap nuclease-polymerase constructs of Table 2 to functional as a polymerase was evaluated using an ffC incorporation assay depicted in FIG. 15A. In this assay, various flap nuclease-polymerase constructs were used to incorporate an ffC nucleotide into a double hairpin template that included two uracils and a Cy5 label nucleotide (star; emits red light). The USER enzyme was then added to generate a gap at the location of the uracil and release the portion of the DNA that includes the Cy5 label and the newly incorporated ffC nucleotide. An agar gel was used to analyze the results. FIG. 15B shows the results (1 = BstFL_GS_Pol(X); 2 = BstFL_Pol(X); 3 = BstFL Helix Pol(X); 4 = BstTR_Helix_Pol(X); 5 = BstTR_GS_Pol(X); 6 = FEN_GS_Pol(X); 7 = FEN_TaqL_Pol(X); 8 = FEN_Helix_Pol(X); 9 = GAK_GS _Pol(X); 10 = GAK_TaqL_Pol(X); 11 = GAK_Helix_Pol(X); 12 = GAN_GS_Pol(X); 13 = GAN_TaqL_Pol(X); 14 = GAN_Helix_Pol(X); 15 = TaqFL_GS_Pol(X); 16 = TaqFL_Pol(X); 17 = TaqFL_Helix_Pol(X); 18 = TaqTR_Helix_Pol(X); 19 = TaqTR_GS_Pol(X)). All constructs were able to efficiently incorporate ffC into the template. The Bst constructs showed evidence of degraded product.

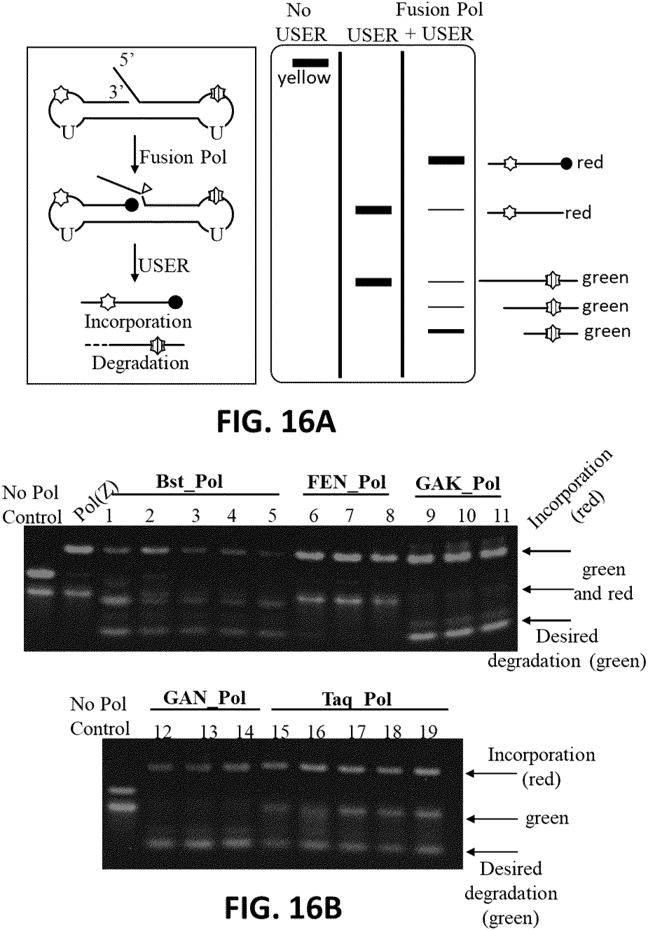

The ability of the flap nuclease-polymerase constructs of Table 2 to function as a polymerase and a flap nuclease was evaluated using a nick translation assay. In this assay, various flap nuclease-polymerase constructs were used to incorporate a ffC nucleotide into a double hairpin template that included a 10 nucleotide flap, two uracils, a Cy5 labeled nucleotide (open star; emits red light) and an iFluoro labeled nucleotide (striped star; emits green light). The USER enzyme (available from New England Biolabs, Ipswich, MA; see also Lindhal, T., Ljungquist, S., Siegert, W., Nyberg, B. and Sperens, B. (1977). J. Biol. Chem. 252, 3286-3294; Lindhal, T. (1982). Annu. Rev. Biochem.. 51, 61-64.; Melamede, R.J., Hatahet, Z., Kow, Y.W., Ide, H. and Wallace, S.S. (1994). Biochemistry. 33, 1255-1264; and Jiang, D., Hatahet, Z., Melamede, R.J., Kow, Y.W. and Wallace, S.S. (1997). J. Biol. Chem. 272, 32230-32239). was then added to generate a gap at the location of both uracils creating three pieces of DNA; a piece that includes the Cy5 labeled nucleotide and the 3′ region of the template; a piece that includes the iFluoro labeled nucleotide and the 5′ region; and a piece that include the region between the uracils (FIG. 16A). An agar gel was then used to analyze the results. Successful incorporation is characterized by the highest molecular weight red band. The degree of nicking is characterized by the molecular weight of the green band. The lower the molecular weight, the greater the number of bases removed from the 5′ flap. The results are shown in FIG. 16B (1 = BstFL_GS_Pol(X); 2 = BstFL_Pol(X); 3 = BstFL_Helix_Pol(X); 4 = BstTR_Helix_Pol(X); 5 = BstTR_GS_Pol(X); 6 = FEN_GS_Pol(X); 7 = FEN_TaqL_Pol(X); 8 = FEN_Helix_Pol(X); 9 = GAK_GS_Pol(X); 10 = GAK_TaqL_Pol(X); 11 = GAK_Helix_Pol(X); 12 = GAN_GS_Pol(X); 13 = GAN_TaqL_Pol(X); 14 = GAN_Helix_Pol(X); 15 = TaqFL_GS_Pol(X); 16 = TaqFL_Pol(X); 17 = TaqFL_Helix_Pol(X); 18 = TaqTR_Helix_Pol(X); 19 = TaqTR_GS_Pol(X)). All fusion constructs were able to efficiently incorporate the ffC into the template. All fusions except FEN1 constructs were able to effectively remove the flap.

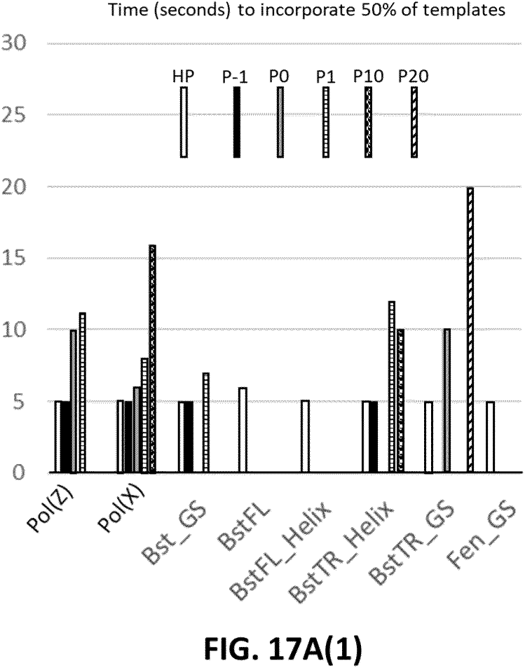

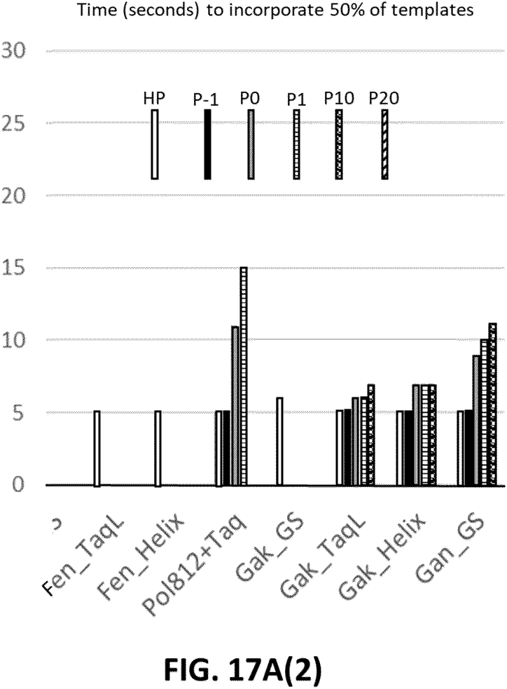

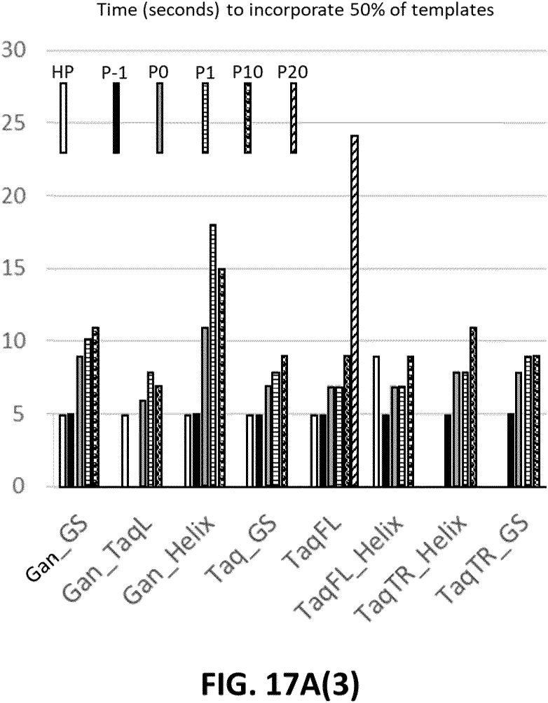

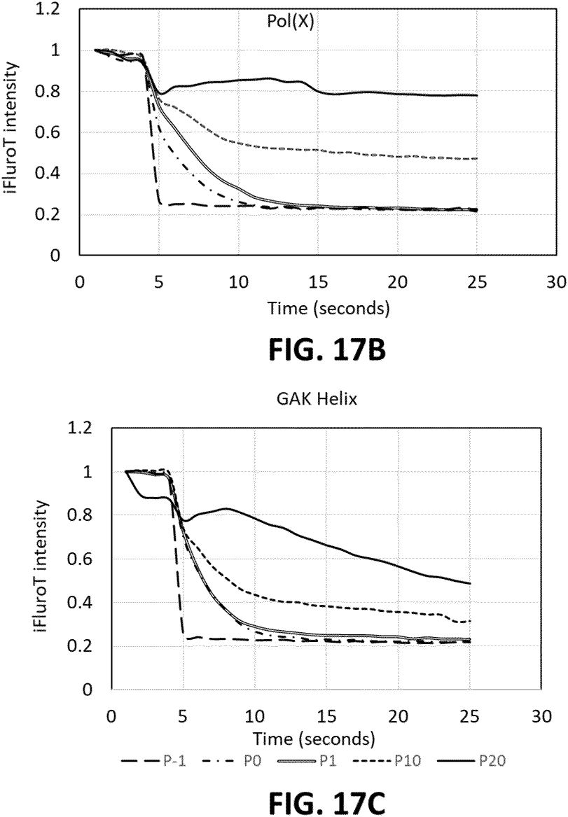

The FRET kinetic assay (FIG. 14A) was used to assess the rate of nucleotide incorporation in the presence of varying flap lengths for the fusions in Table 2 (Hairpin constructs ; P-1 = 1 nt gap, no flap; P0 = nick, no flap; P1 = 1 nt flap; P10 = 10 nt flap; P20 = 20 nt flap). FIGS. 17A-17C show the results. All of the GAK and TAQ fusions as well as the majority of the GAN fusions were able to more efficiently incorporate ffc-Cy5 in the presence of a flap as compared to Pol(X) alone. The GAK_Helix_Pol(X) fusion showed the best performance, nearly eliminating the progressive worsening of incorporation speed with growing flap size up to a 10 nucleotide flap.

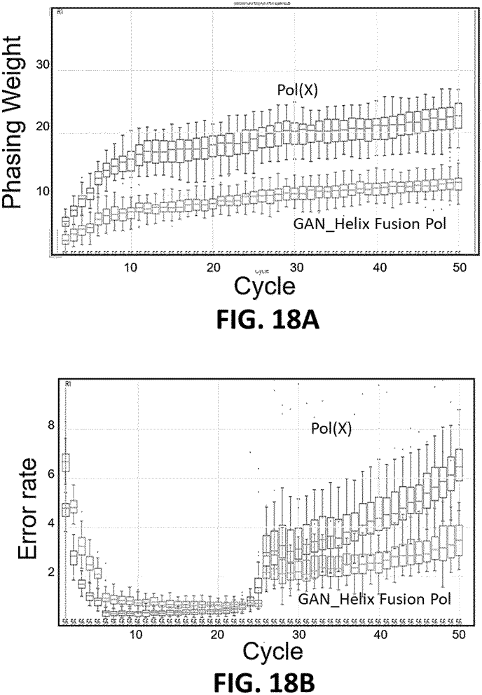

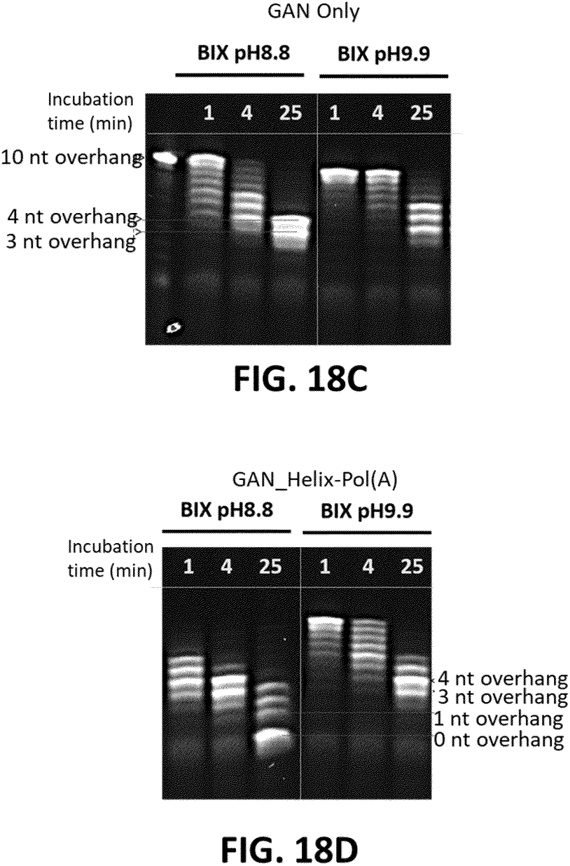

GAN only and the GAN_Helix_Pol(A) construct were compared for their ability to cleave (degrade) a 10 nucleotide flap length from double hairpin template. 0.15 µM of the flap nuclease or flap nuclease fusion was incubated with 0.2 µM amount of the double hairpin template at 50° C. for various time lengths in mixture that included 4 mM MgSO₄ (BIX = 50 mM glycine, 50 mM NaCl, 0.2% CHAPS, 4 mM MgSO4, 1 mM EDTA, at pH 8.8 or 9.9). Two different pH values were tested (8.8 and 9.9). The results are shown in FIGS. 18C and 18D. GAN alone can degrade the 10 nucleotide (nt) flap but degradation stops when an flap length of 3 or 4 nt is reached (18A). In contrast, the GAN_Helix_Pol(A) fusion can completely degrade the 10 nt flap down to a zero nt flap (18D). For both GAN alone and the GAN_Helix_Pol(A) construct, pH 8.8 gave the best results.

The GAN_Helix_Pol(X) fusion error rate and phasing rate were evaluated using a MiniSeq protocol. The results are shown in FIGS. 18A-B. The GAN_Helix-Pol(X) fusion showed lower phasing weights at all cycles compared to Pol(X) alone. Additionally, the GAN_Helix_Pol(X) fusion showed lower error rates than the Pol(X) alone at higher cycle numbers (cycles 30 through 50).

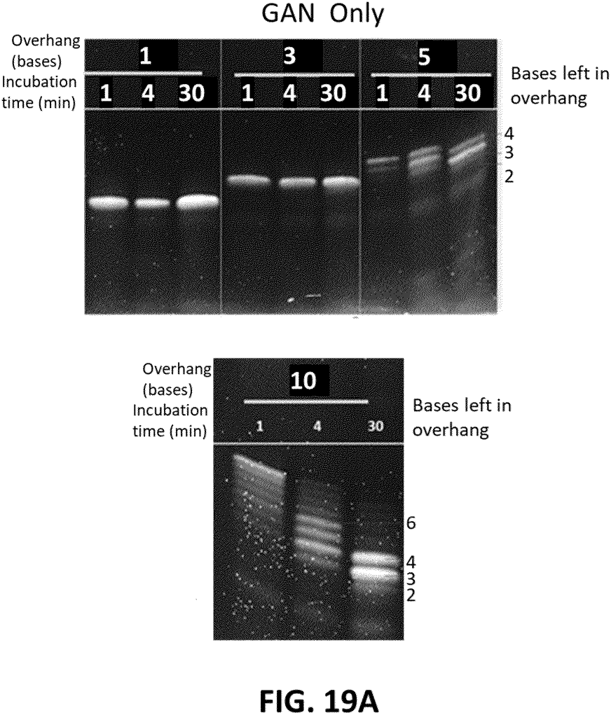

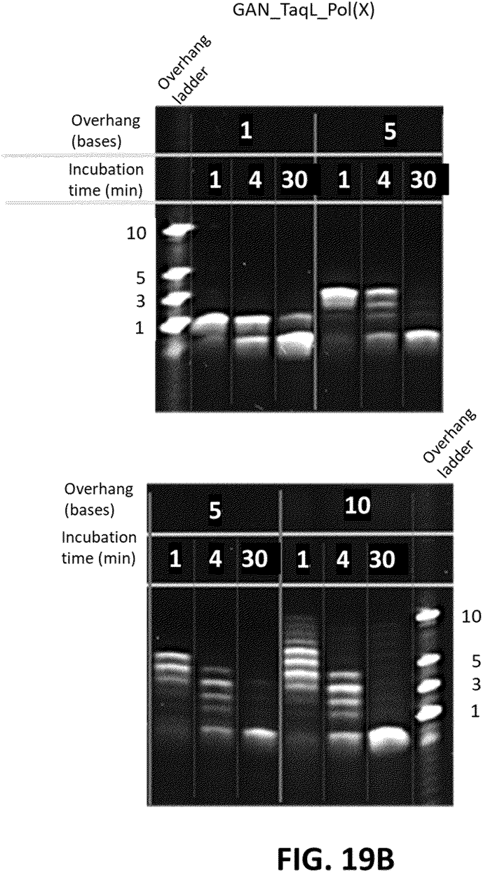

A GAN nuclease and a GAN-Pol(X) fusion linked with a TAQ linker (GAN_TaqL-Pol(X)) were evaluated for their ability to cleave varying flap lengths (1 nt, 3 nt, 5 nt, or 10 nt) from double hairpin template. GAN or the GAN_TaqL-Pol(X) fusion were incubated at 50° C. with the double hairpin templates for various amounts of time (1 min, 4 min, or 30 min). The results are shown in FIGS. 19A and 19B. When GAN is used alone (19A), cleavage does not proceed or is highly reduced once a flap length of 2 or 3 nucleotides is reached. When the fusion is used, cleavage continues until there is no flap (19B).

The error rate of a GAN_TaqL-Pol(A) fusion with dsSurfSeq was assessed relative to the error rate of a Pol(A) alone with ssSurfSeq, Pol(A) alone with dsSurfSeq, Pol(A) used with one times the amount of GAN (Pol(A)+1X GAN) with dsSurfSeq, and Pol(A) used with four times the amount GAN (Pol(A)+4X GAN) with dsSurfSeq. 1X GAN = 1.2 uM, 4X GAN = 4.8 uM, Pol(A) and GAN_TaqL-Pol(A) were used at 1.33 uM.

Assay was performed on an iSeq with ssSurfSeq performed as shown in FIG. 3A and dsSurfSeq performed as shown in FIG. 4. SBS was performed using various methods such as described in U.S. Pat. No. 11,293,061B2 and U.S. Pat. Pub. No. US 2021/0403500A1, with incorporation time extended to 2 minutes.

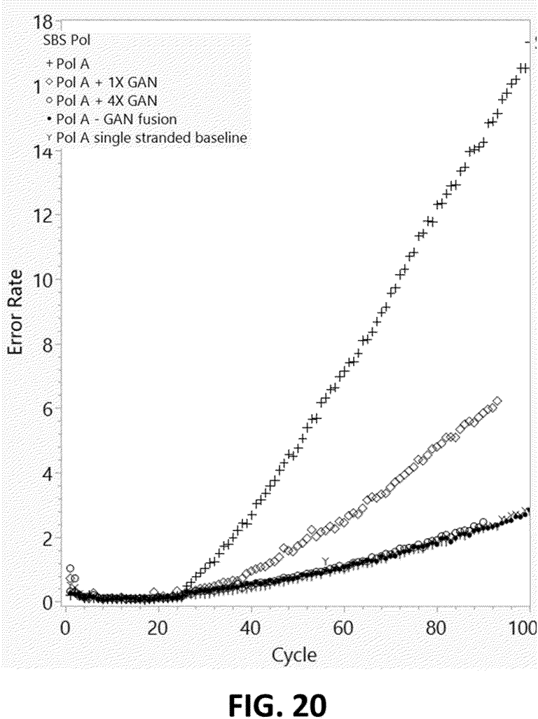

The results are shown in FIG. 20. Pol(A)+1X GAN reduced error rate relative to Pol(A) alone, but not to the level of the ssSurfSeq baseline. Pol(A)+4X GAN reduced error to the level of the ssSurfSEq baseline. The GAN_TaqL-Pol(A) fusion also reduced error to the level of the ssSurfSeq baseline, indicating that a lower concentration of GAN is required when it is fused to Pol(A) compared to when it is mixed with Pol(A) but not fused to it.

### Example 4: Evaluation of dsSurfSeq to Accurately Read Sequences That Include High G Content

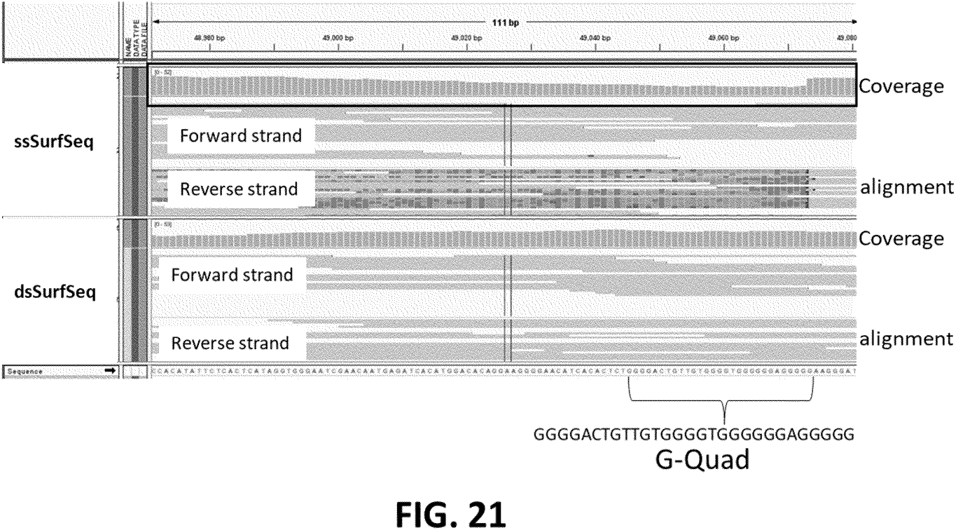

Sequencing performance at a known G-quadruplex region was compared with ssSurfSeq and dsSurfSeq. Assay was performed on an iSeq with ssSurfSeq performed as shown in FIG. 3A and dsSurfSeq performed as shown in FIG. 4. SBS was performed using various methods such as described in U.S. Pat. No. 11,293,061B2 and U.S. Pat. Pub. No. US 2021/0403500A1, with incorporation time extended to 2 minutes. Nicking with GAN_TaqL-Pol(A) fusion was performed as a separate step after incorporation step. Nicking time was 30s per SBS cycle. Sequencing performance at a region containing a known G-quadruplex is shown in FIG. 21. This is a plot prepared using Integrative Genomics Viewer from the Broad Institute.

ssSurfSeq had few errors in the Forward strand, where the DNA template strand does not contain a G-quadruplex, but a large number of errors (darker colored bases) in the Reverse strand, which contains a G-quadruplex. dsSurfSeq had no errors in either the Forward or Reverse strand, demonstrating the ability of double stranded sequencing to remove the effect of G-quadruplexes on sequencing performance.

### Example 5: Evaluation of dsSurfSeq to Protect From Signal Decay Due to Increased Laser Dosage

Signal decay at a range of laser dosages was compared for ssSurfSeq and dsSurfSeq. Assay was performed on a NextSeq2000 with ssSurfSeq performed as shown in FIG. 3A and dsSurfSeq performed as shown in FIG. 4. SBS was performed as described in U.S. Pat. No. 11,293,061B2; U.S. Pat. Pub. No. US 2021/0403500A1; or according to manufactures instructions for NextSeq 1000/2000 chemistry (Illumina Inc., San Diego, CA) . dsSurfSeq used GAN_TaqL-Pol(A), ssSurfSeq used Pol(A). Incorporation time was extended to 2 minutes.

Laser dosage of the blue (450 nm) and green (525 nm) lasers were varied for different tiles of the same flowcell from 1 ms blue, 1 ms green (‘OX’) to 126 ms blue, 225 ms green (‘10X’). Laser power was 2000 mW for blue and 1290 mW for green lasers throughout.

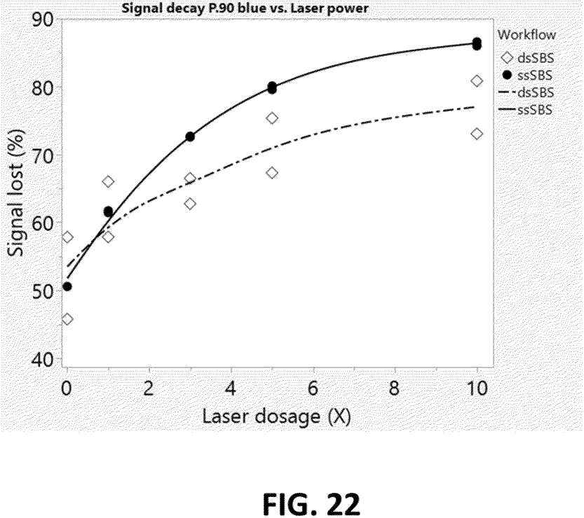

FIG. 22 shows the percentage drop in signal intensity from the beginning of the run to the end for various laser powers for dsSurfSeq and ssSurfSeq. Increase in signal decay with higher laser dosages was less severe for dsSurfSeq than with ssSurfSeq. This demonstrates the ability of double stranded sequencing to protect from laser damage.

A number of embodiments have been described. Nevertheless, it will be understood that various modifications may be made. Accordingly, other embodiments are within the scope of the following claims.

### SEQUENCE LISTING FREE TEXT

SEQ ID NO. 1: P5
TTTTTTTTTT AATGATACGG CGACCACCGA GANCTACAC

where N is uracil.

SEQ ID NO. 2: P7
TTTTTTTTTT CAAGCAGAAG ACGGCATACG ANAT

where N is 8-oxo-guanine.

SEQ ID NO. 3: P15
TTTTTTAATG ATACGGCGAC CACCGAGANC TACAC

where N is allyl T nucleoside.

SEQ ID NO. 4: P17
TTTTTTNNNC AAGCAGAAGA CGGCATACGA GAT

where N is

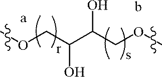

or

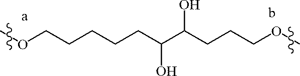

where r is 2, 3, 4, 5, or 6;
s is 2, 3, 4, 5, or 6;
the “a” oxygen is the 3′ hydroxyl oxygen of a first nucleotide and the “b” is the 5′ hydroxyl oxygen of a second nucleotide.

SEQ ID NO. 5: Peptide Linker 1

GGGGSGGGGSGGGGS

SEQ ID NO. 6: Peptide Linker 2

AALGGAAAAAAS

SEQ ID NO. 7: Peptide Linker 3

ALEEAPWPPPWGA

What is claimed is:

1. A sequencing method comprising:
(a) providing a surface, a first surface oligonucleotide bound to the surface at a 5′ end, a second surface primer bound to the surface at a 5′ end and having a free 3′ end, a first polynucleotide template covalently bound to the 3′ end of the first surface oligonucleotide, the first polynucleotide template comprising a free 3′ end, wherein at least a portion of the first polynucleotide template in proximity to the free 3′ end is hybridized to at least a portion of a second surface primer;
(b) sequencing at least a portion of the first polynucleotide template by extending the second surface primer from the free 3′ end using the first polynucleotide template as a template and at least a portion of the second surface primer as a primer, thereby generating a second polynucleotide template covalently bound to the second surface primer and having a free 3′ end, the second polynucleotide template complementary to the first polynucleotide template and complementary to at least a portion of the first surface oligonucleotide in proximity to the free 3′ end, the second polynucleotide template comprising a first read region;
(c) cleaving the first surface oligonucleotide or a 5′ portion of the first polynucleotide template to produce a first surface primer bound to the surface at the 5′ end and having a free 3′ end and to produce a cleaved first polynucleotide template having a free 5′ end and a free 3′ end; and
(d) sequencing at least a portion of the second polynucleotide template by extending the first surface primer from the free 3′ end using the second polynucleotide template as a template and at least a portion of the first surface primer as a primer, thereby generating a third polynucleotide template that is complementary to the first polynucleotide template, the third polynucleotide template comprising a second read region.

2. The method of claim 1, wherein step (a) further comprises:
providing a fourth polynucleotide template complementary to the first polynucleotide template and covalently bound to the 3′ end of the second surface oligonucleotide, the fourth polynucleotide template comprising a free 3′ end, wherein at least a portion of the fourth polynucleotide template in proximity to the free 3′ end is hybridized to at least a portion of the first surface oligonucleotide; and
cleaving the second surface oligonucleotide or a 5′ portion of the fourth polynucleotide template to produce the second surface primer and a cleaved fourth polynucleotide template having a free 5′ end and a free 3′ end.

3. The method of claim 2, wherein cleaving the second surface oligonucleotide or a 5′ portion of the fourth polynucleotide template further comprises:
removing a first excisable base generating a cleaved second surface oligonucleotide; and
generating a hydroxyl at the free 3′ end of the cleaved second surface oligonucleotide to give the second surface primer; or
treating the surface with one or more dihydroxylation reagents to produce the second surface primer.

4. The method of claim 3, wherein the second surface oligonucleotide or the 5′ portion of the fourth polynucleotide template comprises and allyl-dNTP and the method comprises treating the surface with one or more dihydroxylation reagents to produce the second surface primer.

5. The method of claim 4, wherein the one or more dihydroxylation reagents comprises a single reagent comprising OsO₄.

6. The method of claim 1, further comprising providing a cleaved fourth polynucleotide template have a free 5′ end and a free 3′ end, wherein the cleaved fourth polynucleotide template is hybridized to at least a portion of the first polynucleotide template.

7. The method of claim 2, wherein extension of the second surface primer from the free 3′ during sequencing of at least the portion of the first polynucleotide template results in displacement of at least a 5′ portion of the cleaved fourth polynucleotide template from the first polynucleotide template.

8. The method of claim 2, wherein sequencing at least a portion of the first polynucleotide template further comprises:
removing nucleotides and/or polynucleotides from the cleaved fourth polynucleotide template thereby shortening the cleaved fourth polynucleotide template.

9. The method of claim 8, wherein the nucleotides and/or polynucleotides are removed by a flap nuclease.

10. The method of claim 9, wherein a polymerase is used for the sequencing step (d) and wherein the polymerase is operably linked to the flap nuclease in a polymerase-flap nuclease construct.

11. The method of claim 10, wherein the polymerase-flap nuclease construct comprises Taq DNA polymerase, Bst DNA polymerase GAN, FEN1, or a portion thereof that has flap nuclease activity.

12. The method of claim 2, further comprising denaturing the cleaved fourth polynucleotide template from the first polynucleotide template and washing the surface to remove the cleaved fourth polynucleotide template prior to sequencing at least the portion of the first polynucleotide template.

13. The method of claim 1, wherein cleaving the first surface oligonucleotide or a 5′ portion of the first polynucleotide template further comprises:
removing a second excisable base generating a cleaved first surface oligonucleotide; and
generating a hydroxyl at the free 3′ end of the cleaved first surface oligonucleotide to give the second surface primer.

14. The method of claim 1, further comprising denaturing the cleaved first polynucleotide template from the third polynucleotide template and washing the surface to remove the cleaved first polynucleotide template prior to sequencing at least the portion of the second polynucleotide template.

15. The method of claim 1, wherein step (a) further comprising treating the surface with an exonuclease.

16. A kit comprising all reagents needed for sequencing at least the portion the first polynucleotide template and at least the portion of the second polynucleotide template, wherein the kit is free of sequencing primers.

17. The kit of claim 16, wherein the reagents comprise a polymerase and labeled, blocked nucleotides.

18. The kit of claim 16, wherein the reagents comprise a cleavage reagent.

19. The kit of claim 16, further comprising one or more reagents for amplifying template polynucleotides on a surface.

20. The kit of claim 16, wherein the reagents comprise a flap nuclease.


SEQUENCE LISTING (7 sequences)

SEQ ID NO: 1 - DNA, 39 bp, synthetic construct
  source 1..39: mol_type = other DNA
  modified_base 33: mod_base = OTHER; note = N is Uracil
ttttttttttaatgatacggcgaccaccgaganctacac

SEQ ID NO: 2 - DNA, 34 bp, synthetic construct
  source 1..34: mol_type = other DNA
  modified_base 32: mod_base = OTHER; note = N is 8-oxo-guanine
ttttttttttcaagcagaagacggcatacganat

SEQ ID NO: 3 - DNA, 35 bp, synthetic construct
  source 1..35: mol_type = other DNA
  modified_base 29: mod_base = OTHER; note = N is allyl thymine
ttttttaatgatacggcgaccaccgaganctacac

SEQ ID NO: 4 - DNA, 24 bp, synthetic construct
  source 1..24: mol_type = other DNA
  misc_feature 1: note = linked to a TTTTTT sequence through an NNN sequence wherein each N is -O-CH2-CH2-CH2-CH2-CH2-CH(OH)-CH2-CH2-CH2-O- or -O-(CH2)r-CH(OH)-CH(OH)-(CH2)s-O- where r and s are 2, 3, 4, 5, or 6.
caagcagaagacggcatacgagat

SEQ ID NO: 5 - protein, 15 aa, synthetic construct
GGGGSGGGGSGGGGS

SEQ ID NO: 6 - protein, 12 aa, synthetic construct
AALGGAAAAAAS

SEQ ID NO: 7 - protein, 13 aa, synthetic construct
ALEEAPWPPPWGA